(19) United States
(12) Patent Application Publication
(10) Pub. No.: US 2011/0077231 A1
(43) Pub. Date: Mar. 31, 2011

(54) COMPOUNDS

(75) Inventors: Norbert Hauel, Schemmerhofen (DE); Angelo Ceci, Mittelbiberach (DE); Henri Doods, Warthausen (DE); Iris Kauffmann-Hefner, Attenweiler (DE); Ingo Konetzki, Warthausen (DE); Annette Schuler-Metz, Ulm (DE); Walter Rainer, Biberach (DE)

(73) Assignee: BOEHRINGER INGELHEIM INTERNATIONAL GMBH, Ingelheim am Rhein (DE)

(21) Appl. No.: 12/672,465

(22) PCT Filed: Aug. 12, 2008
(86) PCT No.: PCT/EP08/60563

§ 371 Date: Apr. 15, 2010

(30) Foreign Application Priority Data
Aug. 14, 2007 (EP) PCT/EP2007/058408
Feb. 21, 2008 (EP) PCT/EP2008/052157
Feb. 26, 2008 (EP) 08102044.8

(51) Int. Cl. A61K 31/397; C07C 271/24; A61K 31/27; C07D 207/08; A61K 31/40; C07C 311/29; A61K 31/18; C07D 205/04; A61P 29/00

(52) U.S. Cl. 514/210.01; 560/115; 514/484; 548/575; 514/428; 564/89; 514/604; 548/950

(57) ABSTRACT

Novel compounds, with the following being exemplary:

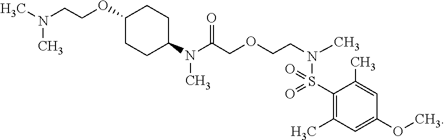

The present invention relates to compounds of general formula I

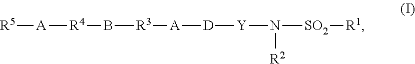

wherein A¹, A², B, D, Y, R¹, R², R³, R⁴ and R⁵ are defined as in claim 1, the enantiomers, diastereomers, mixtures thereof and the salts thereof, particularly the physiologically acceptable salts thereof with organic or inorganic acids or bases, which have valuable properties, the preparation thereof, the pharmaceutical compositions containing the pharmacologically effective compounds, the preparation thereof and the use thereof.

### DETAILED DESCRIPTION OF THE INVENTION

In the above general formula I in a first embodiment
A¹ denotes —CH₂— or a bond,
A² denotes a bond,
B denotes —O—,
D-Y together denote a group selected from

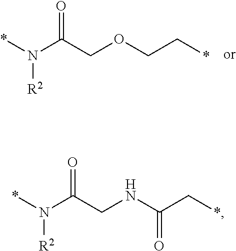

R¹ denotes a group selected from

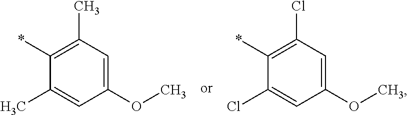

R² denotes H or C₁₋₃-alkyl-, while each methylene group may be substituted by up to two fluorine atoms and each methyl group may be substituted by up to three fluorine atoms, or H₃C—C(O)—,
R³ denotes a C₄₋₆-cycloalkylene group which may be substituted by one, two or three groups R^{3.1},
R^{3.1} denotes —CH₃, —C₂H₅, iso-propyl, tert-butyl, —OH, F, Cl, Br, I,
R⁴ denotes C₁₋₄-alkylene,
R⁵ denotes H₂N, C₁₋₄-alkyl-NH, (C₃₋₆-cycloalkyl)-NH, (C₁₋₄-alkyl)₂N, (C₁₋₄-alkyl)(C₃₋₆-cycloalkyl)N or a 4- to 7-membered saturated heterocyclic ring mono- or disubstituted by R^{5.1}, while the groups R^{5.1} in each case may be identical or different, and
R^{5.1} denotes H, F, Cl, Br, I, C₁₋₃-alkyl-, HO—, C₁₋₃-alkyl-O, (C₁₋₃-alkyl)₂N or C₁₋₃-alkyl-O—C₂₋₄-alkylene-O—,

the enantiomers, the diastereomers, the mixtures and the salts thereof, particularly the physiologically acceptable salts thereof with organic or inorganic acids or bases.

A second embodiment of the present invention comprises the compounds of the above general formula I, wherein A¹, A², B, D, Y, R¹, R², R⁴ and R⁵ are defined as hereinbefore under the first embodiment and
R³ denotes a C₄₋₆-cycloalkylene group which may be substituted by one, two or three groups R^{3.1}, and
R^{3.1} denotes —CH₃, —C₂H₅, iso-propyl, tert-butyl, —OH, F, Cl, Br or I,

with the proviso that the above-mentioned C₄₋₆-cycloalkylene group is linked in the 1,3 position to the remaining molecule,

the enantiomers, the diastereomers, the mixtures and the salts thereof, particularly the physiologically acceptable salts thereof with organic or inorganic acids or bases.

A third embodiment of the present invention comprises the compounds of the above general formula I, wherein A¹, A², B, D, Y, R², R³, R⁴ and R⁵ are defined as mentioned hereinbefore in the first or second embodiment and
R¹ denotes the group

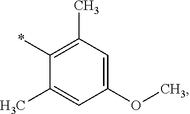

the enantiomers, the diastereomers, the mixtures and the salts thereof, particularly the physiologically acceptable salts thereof with organic or inorganic acids or bases.

A fourth embodiment of the present invention comprises the compounds of the above general formula I, wherein A¹, A², B, D, Y, R², R³, R⁴ and R⁵ are defined as mentioned hereinbefore in the first or second embodiment and
R¹ denotes the group

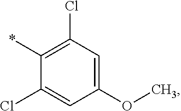

the enantiomers, the diastereomers, the mixtures and the salts thereof, particularly the physiologically acceptable salts thereof with organic or inorganic acids or bases.

A fifth embodiment of the present invention comprises the compounds of the above general formula I, wherein A¹, A², B, D, Y, R¹, R², R³ and R⁵ are defined as mentioned hereinbefore in the first, second, third or fourth embodiment and
R⁴ denotes —CH₂—CH₂—,

the enantiomers, the diastereomers, the mixtures and the salts thereof, particularly the physiologically acceptable salts thereof with organic or inorganic acids or bases.

A sixth embodiment of the present invention comprises the compounds of the above general formula I, wherein A¹, A², B, D, Y, R¹, R², R³ and R⁴ are defined as mentioned hereinbefore in the first, second, third, fourth or fifth embodiment and
R⁵ denotes a group selected from

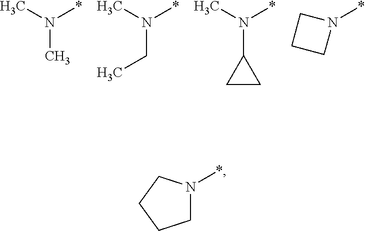

the enantiomers, the diastereomers, the mixtures and the salts thereof, particularly the physiologically acceptable salts thereof with organic or inorganic acids or bases.

A seventh embodiment of the present invention comprises the compounds of the above general formula I, wherein
A¹ denotes —CH₂— or a bond,
A² denotes a bond,
B denotes —O—,
D-Y together denote a group selected from

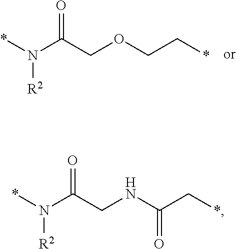

R¹ denotes a group selected from

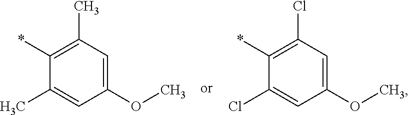

R² denotes H or C₁₋₃-alkyl, while each methylene group may be substituted by up to two fluorine atoms and each methyl group may be substituted by up to three fluorine atoms, or also H₃C—C(O),
R³ denotes a C₄₋₆-cycloalkylene group,
R⁴ denotes —CH₂—CH₂— and
R⁵ denotes a group selected from

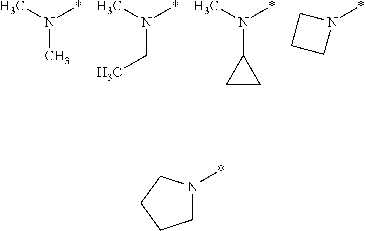

the enantiomers, the diastereomers, the mixtures and the salts thereof, particularly the physiologically acceptable salts thereof with organic or inorganic acids or bases.

The following are mentioned as examples of particularly preferred compounds of the above general formula I:

| Example | Structure |
|---|---|
| (1) | 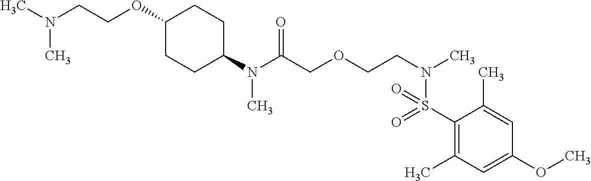 |
| (2) | 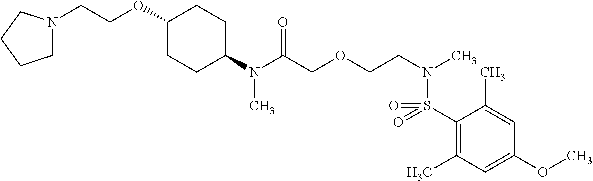 |
| (3) | 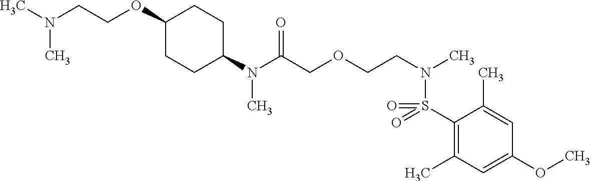 |
| (4) | 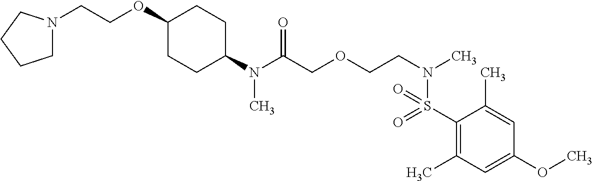 |
| (5) | 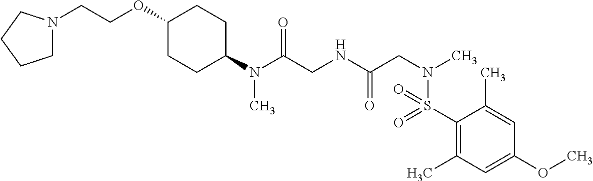 |
| (6) | 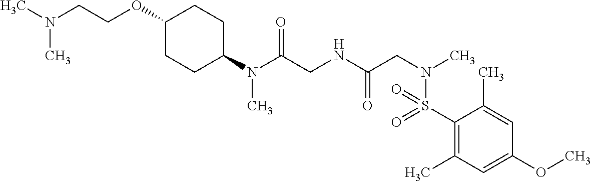 |
| (7) | 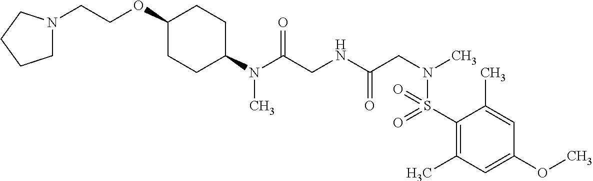 |
| (8) | 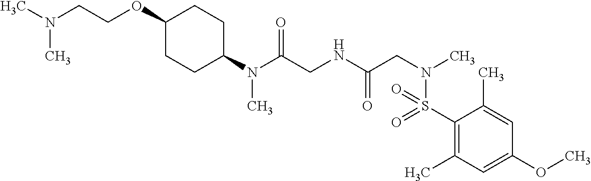 |
| (9) | 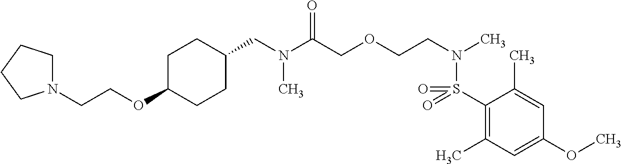 |
| (10) | 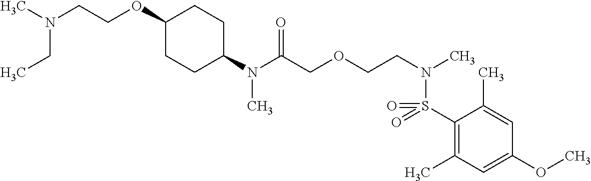 |
| (11) | 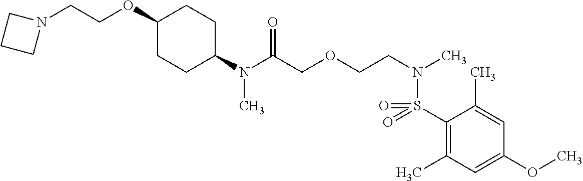 |
| (12) | 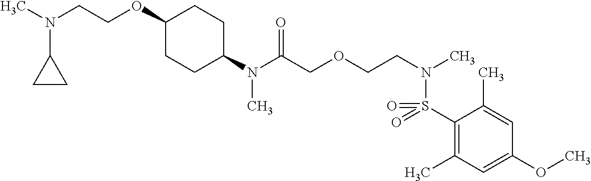 |
| (13) | 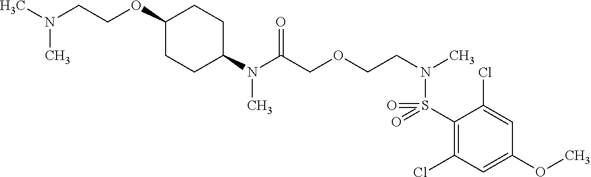 |
| (14) | 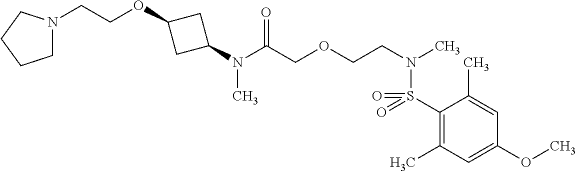 |
| (15) | 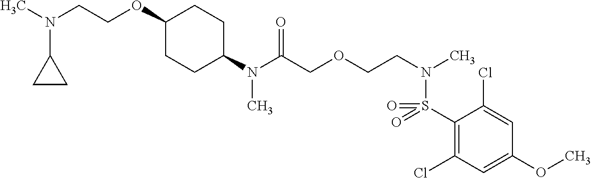 |
| (16) | 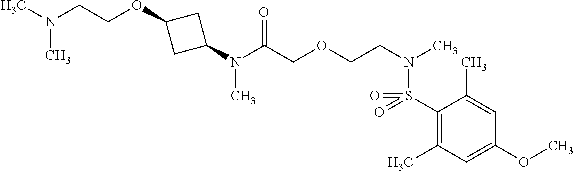 |
| (17) | 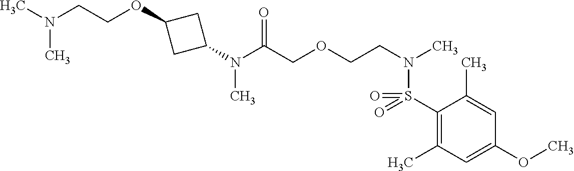 |
| (18) | 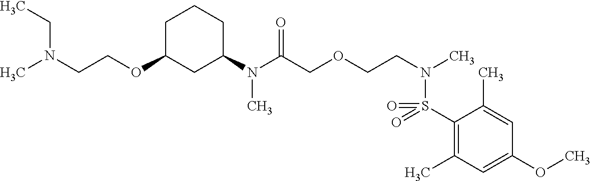 |
| (19) | 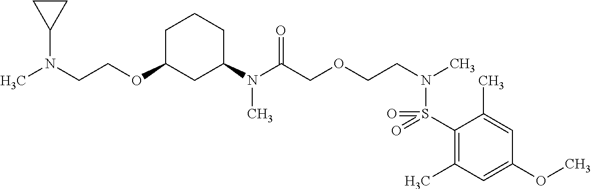 |
| (20) | 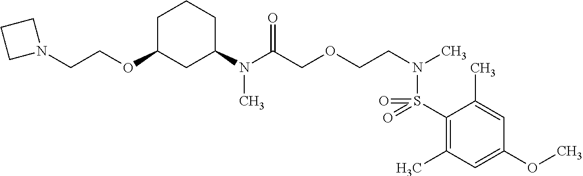 |
| (21) | 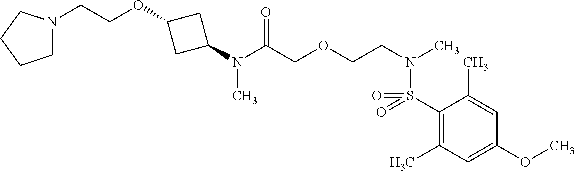 |
| (22) | 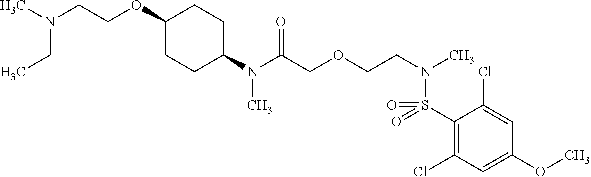 |
| (23) | 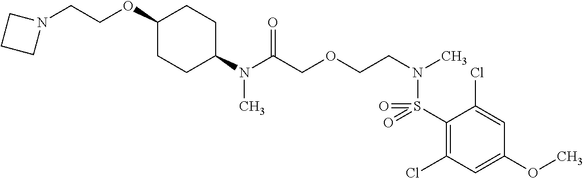 |

the enantiomers, the diastereomers, the mixtures and the salts thereof, particularly the physiologically acceptable salts thereof with organic or inorganic acids or bases.

The following are mentioned as examples of most particularly preferred compounds of the above general formula I:

| Example | Structure |
|---|---|
| (1) | 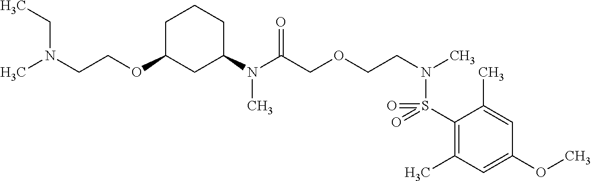 |
| (2) | 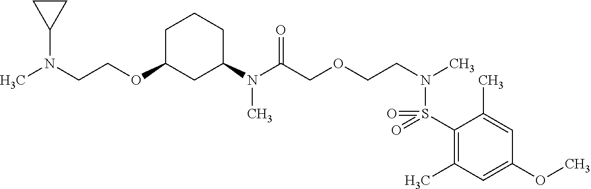 |
| (3) | 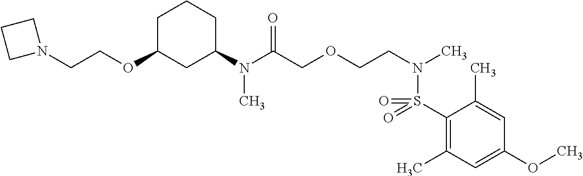 |
| (4) | 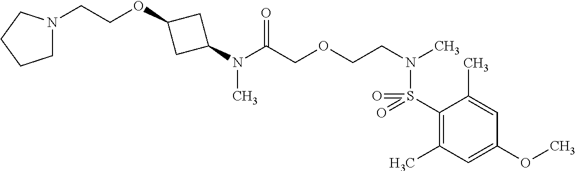 |
| (5) | 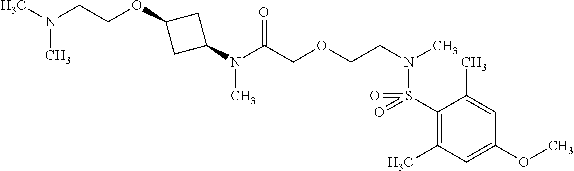 |
| (6) | 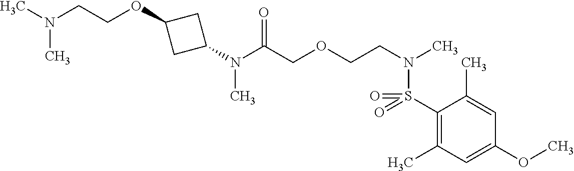 |
| (7) | 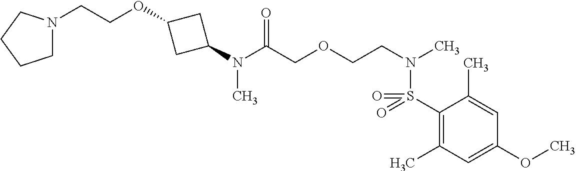 |

the enantiomers, the diastereomers, the mixtures and the salts thereof, particularly the physiologically acceptable salts thereof with organic or inorganic acids or bases.

### Terms and Definitions Used

Unless otherwise stated, all the substituents are independent of one another. If for example there are a plurality of C₁₋₆-alkyl groups as substituents in one group, in the case of three C₁₋₆-alkyl substituents, independently of one another, one may represent methyl, one n-propyl and one tert-butyl.

Within the scope of this application, in the definition of possible substituents, these may also be represented in the form of a structural formula. If present, an asterisk (*) in the structural formula of the substituent is to be understood as being the linking point to the rest of the molecule.

The subject-matter of this invention also includes the compounds according to the invention, including the salts thereof, wherein one or more hydrogen atoms, for example one, two, three, four or five hydrogen atoms, are replaced by deuterium.

By the term “C₁₋₃-alkyl” (including those which are part of other groups) are meant alkyl groups with 1 to 3 carbon atoms and by the term “C₁₋₄-alkyl” are meant branched and unbranched alkyl groups with 1 to 4 carbon atoms. Examples include: methyl, ethyl, n-propyl, iso-propyl, n-butyl, iso-butyl, sec-butyl and tert.butyl. The following abbreviations may also optionally be used for the above-mentioned groups: Me, Et, n-Pr, i-Pr, n-Bu, i-Bu, t-Bu, etc. etc. Unless stated otherwise, the definitions propyl and butyl include all the possible isomeric forms of the respective groups. Thus, for example, propyl includes n-propyl and iso-propyl, butyl includes iso-butyl, sec-butyl and tert-butyl, etc. Moreover, the terms mentioned above also include those groups wherein each methylene group may be substituted by up to two fluorine atoms and each methyl group may be substituted by up to three fluorine atoms.

By the term “C₁₋₄-alkylene” (including those that are part of other groups) are meant branched and unbranched alkylene groups with 1 to 4 carbon atoms and by the term “C₂₋₄-alkylene” are meant branched and unbranched alkylene groups with 2 to 4 carbon atoms. Examples include: methylene, ethylene, ethane-1,1-diyl, propylene, propane-2,2-diyl, 1-methylethylene, butylene, 1-methylpropylene, 1,1-dimethylethylene, 1,2-dimethylethylene. Unless stated otherwise, the definitions propylene and butylene include all the possible isomeric forms of the same number of carbons. Thus, for example, propyl also includes 1-methylethylens and butylene includes 1-methylpropylene, 1,1-dimethylethylene, 1,2-dimethylethylene.

Moreover the definitions mentioned previously also include those groups wherein each methylene group may be substituted by up to two fluorine atoms.

By the term “C₃₋₅-cycloalkyl” (including those which are part of other groups) are meant cyclic alkyl groups with 3 to 5 carbon atoms and by the term “C₃₋₆-alkyl” are meant alkyl groups with 3 to 6 carbon atoms. Examples include: cyclopropyl, cyclobutyl, cyclopentyl or cyclohexyl. Unless otherwise stated, the cyclic alkyl groups may be substituted by one or more groups selected from among methyl, ethyl, iso-propyl, tert-butyl, hydroxy, fluorine, chlorine, bromine and iodine.

By the term “C₄₋₆-cycloalkylene” (including those which are part of other groups) are meant cyclic alkylene groups with 4 to 6 carbon atoms. Examples include: cyclobutylene, cyclopentylene or cyclohexylene. Unless otherwise stated, the cyclic alkylene groups may be substituted by one or more groups selected from among methyl, ethyl, iso-propyl, tert-butyl, hydroxy, fluorine, chlorine, bromine and iodine.

A C₄- or a C₅-cycloalkylene group may be linked in the 1,2 position or in the 1,3 position to the remainder of the molecule, preferably in the 1,3 position. A C₆-cycloalkylene group may be linked in the 1,2 position, in the 1,3 position or in the 1,4 position to the remainder of the molecule, preferably in the 1,3 position.

By the term “saturated heterocyclic rings” are meant four-, five-, six- or seven-membered heterocyclic rings which may contain one or two heteroatoms selected from among oxygen and nitrogen. The ring may be linked to the molecule via a carbon atom or a nitrogen atom. Examples include:

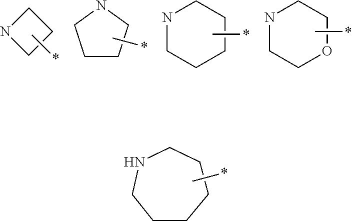

If they contain suitable basic functions, for example amino groups, compounds of general formula I may be converted, particularly for pharmaceutical use, into the physiologically acceptable salts thereof with inorganic or organic acids. Examples of inorganic acids for this purpose include hydrobromic acid, phosphoric acid, nitric acid, hydrochloric acid, sulphuric acid, methanesulphonic acid, ethanesulphonic acid, benzenesulphonic acid or p-toluenesulphonic acid, while organic acids that may be used include malic acid, succinic acid, acetic acid, fumaric acid, maleic acid, mandelic acid, lactic acid, tartaric acid or citric acid. In addition, any tertiary amino groups present in the molecule may be quaternised. Alkyl halides are used for the reaction. According to the invention methyl iodide is preferably used for the quaternisation.

In addition, the compounds of general formula I, if they contain suitable carboxylic acid functions, may if desired be converted into the addition salts thereof with inorganic or organic bases. Examples of inorganic bases include alkali or alkaline earth metal hydroxides, e.g. sodium hydroxide or potassium hydroxide, or carbonates, ammonia, zinc or ammonium hydroxides; examples of organic amines include diethylamine, triethylamine, ethanolamine, diethanolamine, triethanolamine, cyclohexylamine or dicyclohexylamine.

The compounds according to the invention may be present as racemates, provided that they have only one chiral element, but may also be obtained as pure enantiomers, i.e. in the (R) or (S) form.

However, the application also includes the individual diastereomeric pairs of antipodes or mixtures thereof, which are obtained if there is more than one chiral element in the compounds of general formula I, as well as the individual optically active enantiomers of which the above-mentioned racemates are made up.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

### Methods of Preparation

According to the invention the compounds of general formula I are obtained by methods known per se, for example by the following methods:

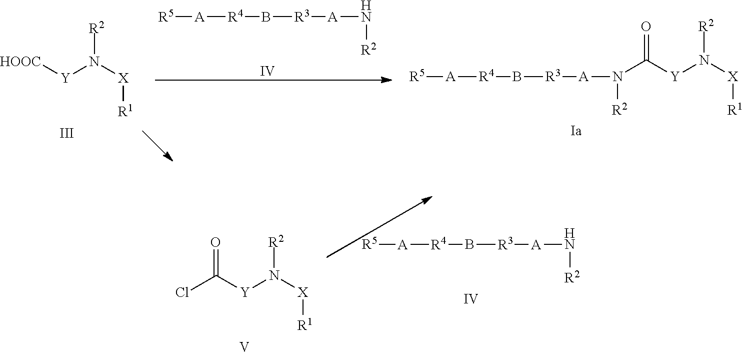

The linking of carboxylic acids of general formula III shown in Scheme 1 wherein all the groups are as hereinbefore defined, with amines of general formula IV, wherein all the groups are as hereinbefore defined, forming carboxylic acid amides of general formula Ia, wherein all the groups are as hereinbefore defined, may be carried out using conventional methods of amide formation.

The coupling is preferably carried out using methods known from peptide chemistry (cf. e.g. Houben-Weyl, Methoden der Organischen Chemie, Vol. 15/2), for example using carbodiimides such as e.g. dicyclohexylcarbodiimide (DCC), diisopropyl carbodiimide (DIC) or ethyl-(3-dimethylaminopropyl)-carbodiimide, O-(1H-benzotriazol-1-yl)-N,N—N′,N′-tetramethyluronium hexafluorophosphate (HBTU) or tetrafluoroborate (TBTU) or 1H-benzotriazol-1-yl-oxy-tris-(dimethylamino)-phosphonium hexafluorophosphate (BOP). By adding 1-hydroxybenzotriazole (HOBt) or 3-hydroxy-4-oxo-3,4-dihydro-1,2,3-benzotriazine (HOObt) the reaction speed can be increased. The couplings are normally carried out with equimolar amounts of the coupling components as well as the coupling reagent in solvents such as dichloromethane, tetrahydrofuran, acetonitrile, dimethyl formamide (DMF), dimethyl acetamide (DMA), N-methylpyrrolidone (NMP) or mixtures thereof and at temperatures between −30° C. and +30° C., preferably −20° C. and +25° C. If necessary, N-ethyl-diisopropylamine (Hünig base) is preferably used as an additional auxiliary base.

An alternative method of attachment consists in converting a carboxylic acid of general formula III, wherein all the groups are as hereinbefore defined, into a carboxylic acid chloride of general formula V, wherein all the groups are as hereinbefore defined, and subsequent reaction with an amine of general formula IV, wherein all the groups are as hereinbefore defined. The synthesis of a carboxylic acid chloride of general formula V is carried out using methods known from the literature (see e.g. Houben-Weyl, Methoden der Organischen Chemie, vol. E5/1).

The carboxylic acids of general formula III used as starting materials, wherein all the groups are as hereinbefore defined, are obtained using methods known per se from the literature, for example by the methods of synthesis shown in Schemes 2 to 7.

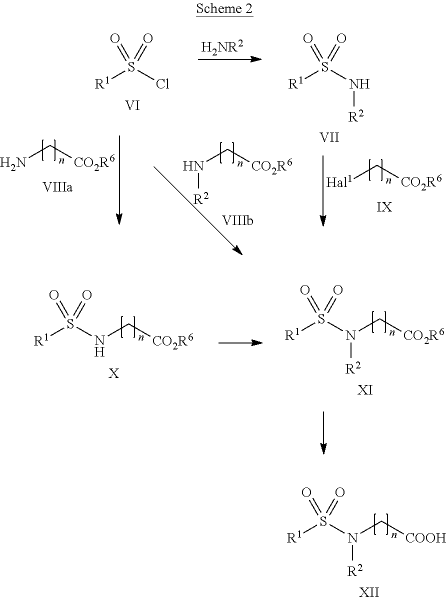

The sulphonic acid chlorides of general formula VI, wherein R¹ is as hereinbefore defined, are either known from the literature or commercially obtainable. They are reacted under standard reaction conditions with an amine of general formulae H₂N—R², VIIIa or VIIIb to obtain sulphonic acid amides of general formulae VII, X or XI, wherein R¹ and R² are hereinbefore defined and n denotes a number 1, 2, 3 or 4 and R⁶ denotes a C₁₋₆-alkyl group. The reaction is optionally carried out in the presence of a base such as triethylamine, DIPEA or pyridine and an inert solvent such as dichloromethane or tetrahydrofuran at a temperature of 0° C. to 100° C. with a typical reaction time of one to 24 hours.

The reaction of the sulphonic acid amides of general formula VII with a halide of general formula IX, wherein Hal¹ denotes chlorine or bromine, is carried out using methods known from the literature, for example with the aid of a base such as potassium or sodium carbonate in dimethylformamide or tetrahydrofuran at 0° C. to 100° C.

The hydrolysis of the carboxylic acid esters of general formula XI, wherein R¹ and R² are as hereinbefore defined, n denotes a number 1, 2, 3 or 4 and R⁶ denotes a C₁₋₃-alkyl group, to obtain carboxylic acids of general formula XII, wherein R¹ and R² are as hereinbefore defined and n denotes a number 1, 2, 3 or 4 and R⁶ denotes a C₁₋₃-alkyl group, is carried out under known conditions, for example with lithium or sodium carbonate and water in methanol and/or tetrahydrofuran.

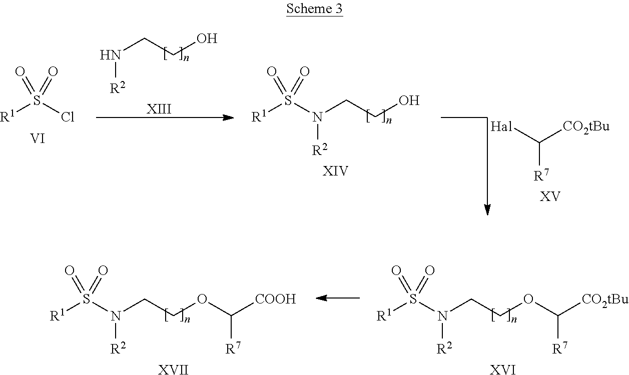

The preparation of sulphonic acid amides of general formula XIV is carried out as described under Scheme 2.

The alkylation of the hydroxyl function of the sulphonic acid amides of general formula XIV, wherein R¹ and R² are as hereinbefore defined with the proviso that R² does not denote a hydrogen atom, and n denotes a number 1, 2, 3 or 4 and R⁶ denotes a C₁₋₃-alkyl group, is carried out under reaction conditions known from the literature, for example under 2-phase conditions using a phase transfer catalyst in the presence of a strong inorganic base such as sodium hydroxide solution or potassium hydroxide solution and in an inert solvent such as toluene at 0° C. to 100° C.

The cleaving of the tert-butylester of general formula XVI, wherein R¹ and R² are as hereinbefore defined, n denotes a number 1, 2, 3 or 4 and R⁶ denotes a C₁₋₃-alkyl group and R⁷ denotes a hydrogen atom or a C₁₋₃-alkyl group, is carried out using methods known from the literature (see e.g. Philip J. Kocieński, Protecting Groups, 3rd Edition, 2005, published by Georg Thieme).

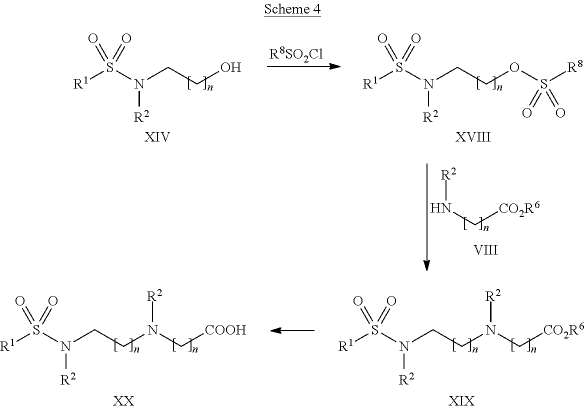

The sulphonation of the hydroxyl function of a compound of general formula XIV, wherein R¹ and R² are as hereinbefore defined, with the proviso that R² does not denote a hydrogen atom, and n denotes a number 1, 2, 3 or 4 and R⁶ denotes a C₁₋₃-alkyl group, with a sulphonic acid chloride of general formula R⁸SO₂Cl, wherein R⁸ denotes a C₁₋₃-alkyl group or a phenyl group optionally substituted by a C₁₋₃-alkyl group, to form compounds of general formula XVIII, wherein all the groups are as hereinbefore defined, is carried out under standard reaction conditions, typically in the presence of a base such as DMAP and/or pyridine and an inert solvent such as dichloromethane or THF at −5° C. to 35° C. A liquid base such as pyridine may be used as the base and solvent simultaneously.

The subsequent alkylation of the amines of general formula VII to form compounds of general formula XIX, wherein R¹ and R² are as hereinbefore defined, n denotes a number 1, 2, 3 or 4 and R⁶ denotes a C₁₋₃-alkyl group and R⁶ denotes a C₁₋₆-alkyl group, is conveniently carried out in a solvent such as toluene, chlorobenzene, dimethylformamide, dimethylsulphoxide (DMSO), dichloromethane, acetonitrile or pyridine, for example at temperatures between 0° C. and 150° C. and conveniently in the presence of bases such as pyridine, triethylamine, DIPEA, potassium carbonate, potassium-tert-butoxide or sodium methoxide, the alkylsulphonate serving as the leaving group.

The hydrolysis of the carboxylic acid esters of general formula XIX to form carboxylic acids of general formula XX is carried out as described under Scheme 2.

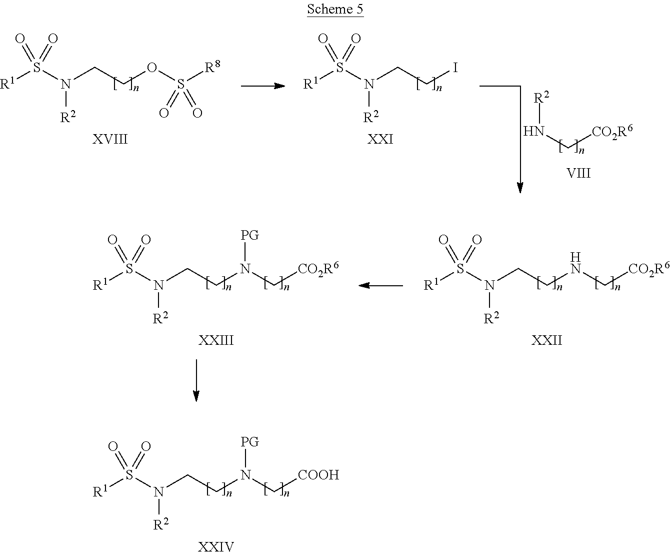

The Finkelstein reaction of compounds of general formula XVIII, wherein R¹ and R² are as hereinbefore defined, n denotes a number 1, 2, 3 or 4 and R⁶ denotes a C₁₋₃-alkyl group and R⁸ denotes a C₁₋₃-alkyl group or a phenyl group optionally substituted by a C₁₋₃-alkyl group, to form halides of general formula XXI, wherein R¹ and R² are as hereinbefore defined and n denotes a number 1, 2, 3 or 4 and R⁶ denotes a C₁₋₃-alkyl group, is carried out under known reaction conditions (see e.g. H. Finkelstein, Berichte der Deutschen Chemischen Gesellschaft 43, 1910, 1528).

The subsequent alkylation of the glycine ester is carried out as described under Scheme 4 (R²≠H).

The amino function in the compounds of general formula XXIII is protected by a conventional protective group PG by known methods. The selected protective group is one which can be cleaved under non-hydrogenolytic conditions. A preferred protective group is the Boc group. An overview of the chemistry of protective groups can be found in Theodora W. Greene and Peter G. M. Wuts, Protective Groups in Organic Synthesis, Second Edition, 1991, published by John Wiley and Sons, and in Philip J. Kocieński, Protecting Groups, 3rd Edition, 2005, published by Georg Thieme.

The cleaving of the carboxylic acid esters of general formula XXIII to form carboxylic acids of general formula XXIV is carried out as described under Scheme 2.

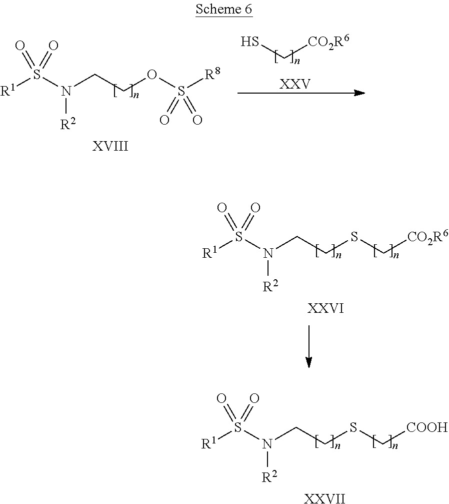

The alkylation of a thiol of general formula XXV, wherein n denotes a number 1, 2, 3 or 4 and R⁶ denotes a C₁₋₆-alkyl group, to obtain compounds of general formula XXVI, wherein R¹ and R² are as hereinbefore defined, n denotes a number 1, 2, 3 or 4 and R⁶ denotes a C₁₋₆-alkyl group, is conveniently carried out in a solvent such as toluene, chlorobenzene, DMF, DMSO, dichloromethane, acetonitrile or pyridine, for example at temperatures between 0° C. and 150° C. and conveniently in the presence of bases such as pyridine, triethylamine, DIPEA, potassium carbonate, potassium-tert-butoxide or sodium methoxide, while the alkylsulphonate serves as leaving group.

The hydrolysis of the carboxylic acid esters of general formula XXVI to form carboxylic acids of general formula XXVII, wherein all the groups are as hereinbefore defined, is carried out as described under Scheme 2.

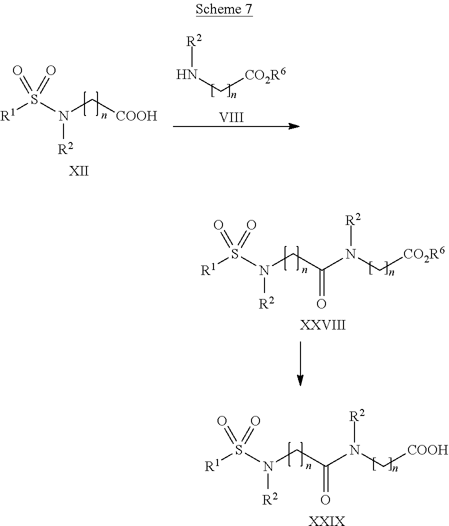

The amide linking of carboxylic acids of general formula XII, wherein R¹ and R² are as hereinbefore defined and n denotes a number 1, 2, 3 or 4, and amino acids of general formula VIII, wherein R¹ and R² are as hereinbefore defined, n denotes a number 1, 2, 3 or 4 and R⁶ denotes a C₁₋₆-alkyl group, to obtain carboxylic acid amides of general formula XVIII, wherein R¹ and R² are as hereinbefore defined, n denotes a number 1, 2, 3 or 4 and R⁶ denotes a C₁₋₆-alkyl group, is carried out as described under Scheme 1.

As mentioned under Scheme 2, the carboxylic acid ester of general formula XXVIII is cleaved to form carboxylic acid of general formula XXIX, wherein R¹ and R² are as hereinbefore defined and n denotes a number 1, 2, 3 or 4.

The amines of general formula IV used as starting materials are either commercially obtainable, or are obtained using methods known per se from the literature, for example by the methods of synthesis represented in Schemes 8 to 12, wherein R^{1.1} is as hereinbefore defined, Hal¹ denotes a chlorine or bromine atom and Hal² denotes a fluorine, chlorine or bromine atom or a group R⁹.

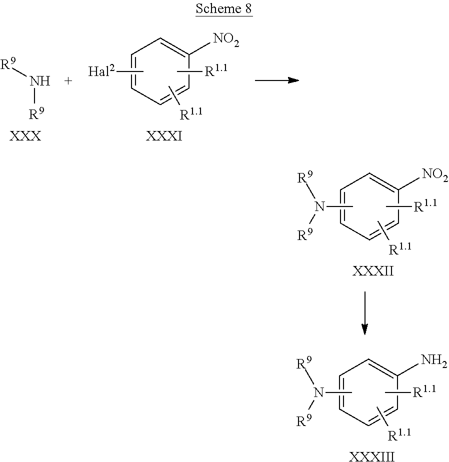

The reaction of an amine of general formula XXX, wherein R⁹ denotes a C₁₋₃-alkyl group, with a halo-nitrobenzene of general formula XXXI, wherein R^{1.1} is as hereinbefore defined and Hal² denotes a fluorine, chlorine or bromine atom or a group R⁹, is carried out using known methods, for example in a solvent such as tetrahydrofuran, dimethylformamide or dimethylsulphoxide and conveniently in the presence of a suitable base such as triethylamine or potassium carbonate, at a temperature of 20° C. to 160° C. If the amine of general formula XXX is liquid, the reaction may also be carried out without a solvent and additional base.

The reduction of the nitro group to form anilines of general formula XXXIII, wherein R^{1.1} is as hereinbefore defined and R⁹ denotes a C₁₋₃-alkyl group, is carried out under standard reaction conditions (see e.g. Richard C. Larock, Comprehensive Organic Transformations, 1989, VCH), preferably under standard conditions of catalytic hydrogenolysis with a catalyst such as palladium on charcoal or Raney nickel in a solvent such as methanol or ethanol.

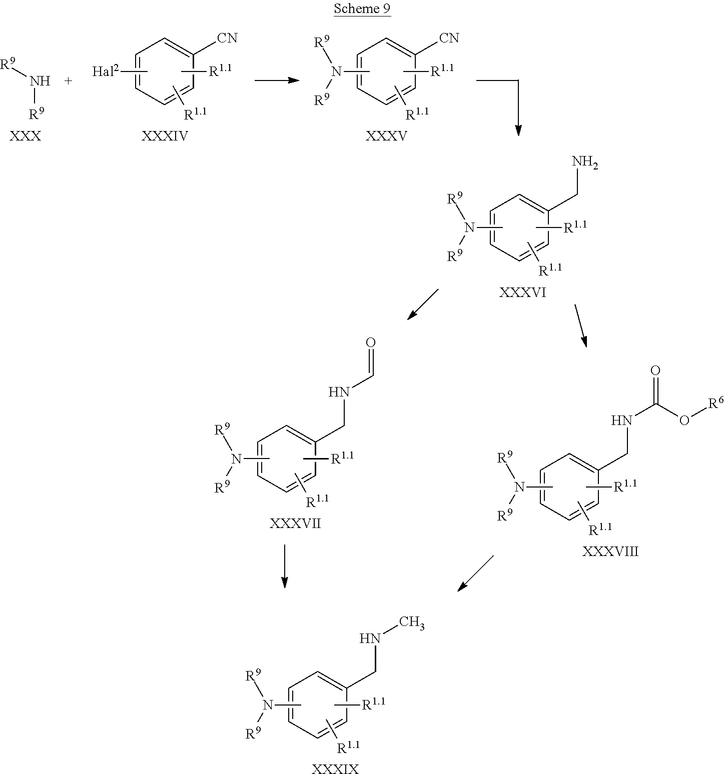

The reaction of compounds of general formulae XXX, wherein R⁹ denotes a C₁₋₃-alkyl group, with a compound of general formula XXXIV, wherein R^{1.1} is as hereinbefore defined and Hal² denotes a fluorine, chlorine or bromine atom or a group R⁹, to obtain compounds of general formula XXXV, wherein R¹¹ is as hereinbefore defined and R⁹ denotes a C₁₋₃-alkyl group, is carried out as described under Scheme 8.

The reduction of a nitrile of general formula XXXV to form an amine of general formula XXXVI, wherein R^{1.1} is as hereinbefore defined and R⁹ denotes a C₁₋₃-alkyl group, may be carried out under standard conditions of catalytic hydrogenolysis with a catalyst such as for example Raney nickel in a solvent such as ammoniacal methanol or ethanol or with a reducing agent such as lithium aluminium hydride or sodium borohydride in a solvent such as tetrahydrofuran, optionally in the presence of aluminium chloride.

The formylation of an amine of general formula XXXVI to obtain a compound of general formula XXXVII, wherein R^{1.1} is as hereinbefore defined and R⁹ denotes a C₁₋₃-alkyl group, is conveniently carried out in a solvent such as dichloromethane, for example at temperatures from 40° C. to 70° C. and in the presence of acetic anhydride and formic acid.

The carbamate formation to obtain compounds of general formula XXXVIII, wherein R^{1.1} is as hereinbefore defined, R⁶ denotes a C₁₋₆-alkyl and R⁹ denotes a C₁₋₃-alkyl group is carried out by known methods, for example with a chloroformic acid ester or Boc-anhydride in the presence of a base such as triethylamine or sodium hydroxide solution and a solvent such as THF or dioxane.

The reduction of the formyl or of the carbamate to obtain compounds of general formula XXXIX, wherein R^{1.1} is as hereinbefore defined and R⁹ denotes a C₁₋₃-alkyl group, is carried out under standard reaction conditions, preferably with a reducing agent such as lithium aluminium hydride and in a solvent such as tetrahydrofuran at a temperature of 50° C. to 100° C.

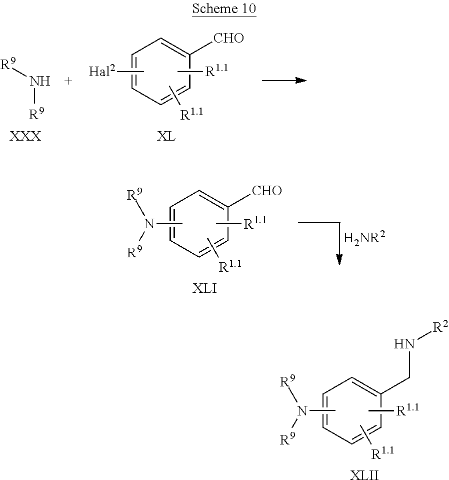

The halogen-nitrogen exchange in compounds of general formulae XXX, wherein R⁹ denotes a C₁₋₃-alkyl group, and XL, wherein R^{1.1} is as hereinbefore defined and Hal² denotes a fluorine, chlorine or bromine atom or a group R⁹, for preparing compounds of general formula XLI, wherein R^{1.1} is as hereinbefore defined and R⁹ denotes a C₁₋₃-alkyl group, is carried out as described under Scheme 8.

The reaction of benzaldehydes of general formula XLI, wherein R^{1.1} is as hereinbefore defined and R⁹ denotes a C₁₋₃-alkyl group, with an amine of general formula H₂NR², wherein R² is as hereinbefore defined, to obtain a compound of general formula XLII, wherein R^{1.1} and R² are as hereinbefore defined and R⁹ denotes a C₁₋₃-alkyl group, is a reductive amination. It is carried out by known methods, for example with a reducing agent such as sodium triacetoxyborohydride, sodium borohydride or sodium cyanoborohydride, conveniently in a solvent such as tetrahydrofuran or dichloromethane, optionally with the addition of acetic acid.

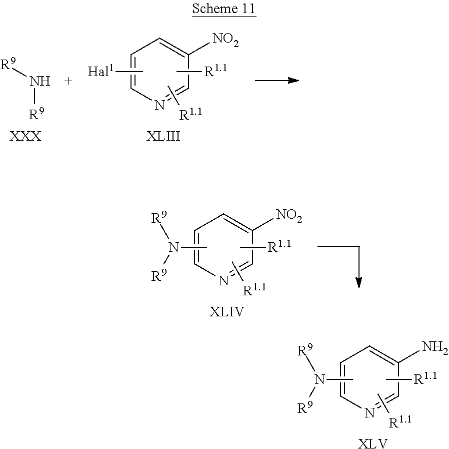

The reaction of an amine of general formula XXX, wherein R⁹ denotes a C₁₋₃-alkyl group, with a halogen-nitropyridine of general formula XLIII, wherein R^{1.1} is as hereinbefore defined and Hal¹ denotes a chlorine or bromine atom, is carried out by known methods, for example in a solvent such as tetrahydrofuran, dichloromethane, methanol or DMSO and conveniently in the presence of a suitable base such as triethylamine, sodium hydroxide solution or potassium carbonate and at a temperature of 20° C. to 100° C.

The subsequent reduction of the nitro group of a compound of general formula XLIV, wherein R^{1.1} is as hereinbefore defined and R⁹ denotes a C₁₋₃-alkyl group, to obtain compounds of general formula XLV, wherein R^{1.1} is as hereinbefore defined and R⁹ denotes a C₁₋₃-alkyl group, is carried out as described under Scheme 8.

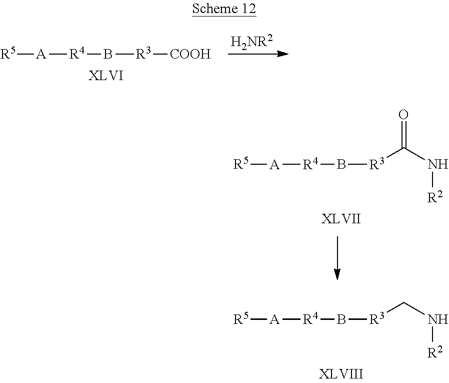

The amide linking of carboxylic acids of general formula XLVI, wherein all the groups are as hereinbefore defined, and amines of general formula H₂NR², wherein R² is as hereinbefore defined, to form carboxylic acid amides of general formula XLVII, wherein all the groups are as hereinbefore defined, is carried out as described under Scheme 1.

The reduction of carboxylic acid amides of general formula XLVII to obtain amines of general formula XLVIII, wherein all the groups are as hereinbefore defined, is carried out under standard reaction conditions, preferably in the presence of a reducing agent such as lithium aluminium hydride and a solvent such as tetrahydrofuran at 40° C. to 100° C.

Description of the Method of hBK1 Receptor Binding

CHO cells expressing the hBK1 receptor are cultivated in Dulbecco's modified medium. The medium from confluent cultures is removed and the cells are washed with PBS buffer, scraped off and isolated by centrifugation. The cells are then homogenized in suspension and the homogenate is centrifuged and resuspended. The protein content is determined and the membrane preparation obtained in this manner is then frozen at −80° C. After thawing, 200 μl of the homogenate (50 to 100 μg of proteins/assay) are incubated at room temperature with 0.5 to 1.0 nM of kallidin (DesArg10, Leu9), [3,4-prolyl-3,43H(N)] and increasing concentrations of the test substance in a total volume of 250 μl for 60 minutes. The incubation is terminated by rapid filtration through GF/B glass fibre filters which had been pretreated with polyethyleneimine (0.3%). The protein-bound radioactivity is measured in a TopCount NXT. Non-specific binding is defined as radioactivity bound in the presence of 1.0 μM of kallidin (DesArg10, Leu9), [3,4-prolyl-3,43H(N)]. The concentration/binding curve is analysed using a computer-assisted nonlinear curve fitting. The Kᵢ which corresponds to the test substance is determined using the data obtained in this manner.

To demonstrate that the compounds of general formula I with different structural elements show good to very good bradykinin-B1-receptor antagonistic effects, the following Table gives the Kᵢ values obtained according to the test method described above. It is pointed out that the compounds were selected for their different structural elements and not in order to emphasise specific compounds:

| Example | Kᵢ [nM] |
|---|---|
| (1) | 53.6 |
| (2) | 15 |

### Indications

By virtue of their pharmacological properties, the novel compounds and their physiologically acceptable salts are suitable for treating diseases and symptoms of diseases caused at least to some extent by stimulation of bradykinin-B1 receptors.

In view of their pharmacological effect the substances are suitable for the treatment of

(a) acute pain such as e.g. toothache, peri- and postoperative pain, traumatic pain, muscle pain, the pain caused by burns, sunburn, trigeminal neuralgia, pain caused by colic, as well as spasms of the gastro-intestinal tract or uterus;

(b) visceral pain such as e.g. chronic pelvic pain, gynaecological pain, pain before and during menstruation, pain caused by pancreatitis, peptic ulcers, interstitial cystitis, renal colic, angina pectoris, pain caused by irritable bowel, non-ulcerative dyspepsia and gastritis, non-cardiac thoracic pain and pain caused by myocardial ischaemia and cardiac infarct;

(c) neuropathic pain such as e.g. painful neuropathies, pain of diabetic neuropathy, AIDS-associated neuropathic pain, pain of lumbago, non-herpes-associated neuralgia, post-zoster neuralgia, nerve damage, cerebro-cranial trauma, pain of nerve damage caused by toxins or chemotherapy, phantom pain, pain of multiple sclerosis, nerve root tears and painful traumatically-caused damage to individual nerves;

(d) inflammatory/pain receptor-mediated pain in connection with diseases such as osteoarthritis, rheumatoid arthritis, rheumatic fever, tendo-synovitis, tendonitis, gout, vulvodynia, damage to and diseases of the muscles and fascia (muscle injury, fibromyalgia), osteoarthritis, juvenile arthritis, spondylitis, gout-arthritis, psoriasis-arthritis, fibromyalgia, myositis, migraine, dental disease, influenza and other virus infections such as colds, systemic lupus erythematodes,

(e) tumour pain associated with cancers such as lymphatid or myeloid leukaemia, Hodgkin's disease, non-Hodgkin's lymphomas, lymphogranulomatosis, lymphosarcomas, solid malignant tumours and extensive metastases;

(f) headache diseases such as e.g. headache of various origins, cluster headaches, migraine (with or without aura) and tension headaches.

The compounds are also suitable for treating

(g) inflammatory changes connected with diseases of the airways such as bronchial asthma, including allergic asthma (atopic and non-atopic) as well as bronchospasm on exertion, occupationally induced asthma, viral or bacterial exacerbation of an existing asthma and other non-allergically induced asthmatic diseases;

chronic obstructive pulmonary disease (COPD) including pulmonary emphysema, acute adult respiratory distress syndrome (ARDS), bronchitis, lung inflammation, allergic rhinitis (seasonal and all year round), vasomotor rhinitis and diseases caused by dust in the lungs such as aluminosis, anthracosis, asbestosis, chalicosis, siderosis, silicosis, tabacosis and byssinosis;

(h) inflammatory phenomena caused by sunburn and burns, oedema after burns trauma, cerebral oedema and angiooedema, intestinal complaints including Crohn's diseases and ulcerative colitis, irritable bowel syndrome, pancreatitis, nephritis, cystitis (interstitial cystitis), uveitis; inflammatory skin diseases (such as e.g. psoriasis and eczema), vascular diseases of the connective tissue, lupus, sprains and fractures;

(i) diabetes mellitus and its effects (such as e.g. diabetic vasculopathy, diabetic neuropathy, diabetic retinopathy) and diabetic symptoms in insulitis (e.g. hyperglycaemia, diuresis, proteinuria and increased renal excretion of nitrite and kallikrein);

(j) neurodegenerative diseases such as Parkinson's disease and Alzheimer's disease;

(k) sepsis and septic shock after bacterial infections or after trauma;

(l) syndromes that cause itching and allergic skin reactions;

(m) osteoporosis;

(n) epilepsy;

(o) damage to the central nervous system;

(p) wounds and tissue damage;

(q) inflammation of the gums;

(r) benign prostatic hyperplasia and hyperactive bladder;

(s) pruritus;

(t) vitiligo;

(u) disorders of the motility of respiratory, genito-urinary, gastro-intestinal or vascular regions and

(v) post-operative fever.

In addition to being suitable as human therapeutic agents, these substances are also useful in the veterinary treatment of domestic animals, exotic animals and farm animals. For treating pain, it may be advantageous to combine the compounds according to the invention with stimulating substances such as caffeine or other pain-alleviating active compounds. If active compounds suitable for treating the cause of the pain are available, these can be combined with the compounds according to the invention. If, independently of the pain treatment, other medical treatments are also indicated, for example for high blood pressure or diabetes, the active compounds required can be combined with the compounds according to the invention.

The following compounds may be used for combination therapy, for example:

Non-steroidal antirheumatics (NSAR): COX-2 inhibitors such as propionic acid derivatives (alminoprofen, benoxaprofen, bucloxic acid, carprofen, fenhufen, fenoprofen, fiuprofen, fiulbiprofen, ibuprofen, indoprofen, ketoprofen, miroprofen, naproxen, oxaprozin, pirprofen, pranoprofen, suprofen, tiaprofenic acid, tioxaprofen), acetic acid derivatives (indomethacin, acemetacin, alcofenac, isoxepac, oxpinax, sulindac, tiopinac, tolmetin, zidometacin, zomepirac) fenamic derivatives (meclofenamic acid, mefenamic acid, tolfenamic acid), biphenyl-carboxylic acid derivatives, oxicams (isoxicam, meloxicam, piroxicam, sudoxicam and tenoxicam), salicylic acid derivatives (acetylsalicylic acid, sulphasalazin, mesalazine, and olsalazine), pyrazolone (apazone, bezpiperylone, feprazone, mofebutazone, oxyphenbutazone, phenylbutazone, propyphenazone and metamizol), and coxibs (celecoxib, valecoxib, rofecoxib, etoricoxib).

Opiate receptor agonists such as e.g. morphine, propoxyphen (Darvon), tramadol, buprenorphine.

Cannabinoid agonists such as e.g. GW-1000, KDS-2000, SAB-378, SP-104, NVP001-GW-843166, GW-842166X, PRS-211375.

Sodium channel blockers such as e.g. carbamazepine, mexiletin, lamotrigin, pregabalin, tectin, NW-1029, CGX-1002.

N-type calcium channel blockers such as e.g. ziconitide, NMED-160, SP1-860.

Serotonergic and noradrenergic modulators such as e.g. SR-57746, paroxetine, duloxetine, clonidine, amitriptyline, citalopram.

Corticosteroids such as e.g. betamethasone, budesonide, cortisone, dexamethasone, hydrocortisone, methylprednisolone, prednisolone, prednisone and triamcinolone. Histamine H1-receptor antagonists such as e.g. bromopheniramine, chloropheniramine, dexchlorpheniramine, triprolidine, clemastine, diphenhydramine, diphenylpyraline, tripelennamine, hydroxyzine, methdilazine, promethazine, trimeprazine azatadine, cyproheptadine, antazoline, pheniramine, pyrilamine, astemizole, terfenadine, loratadine, cetirizine, desloratadine, fexofenadine, levocetirizine.

Histamine H2-receptor antagonists such as e.g. cimetidine, famotidine, and ranitidine. Proton pump inhibitors such as e.g. omeprazole, pantoprazole, esomeprazole.

Leukotriene antagonists and 5-lipoxygenasehemmer such as e.g. zafirlukast, montelukast, pranlukast and zileuton.

Local anaesthetics such as e.g. ambroxol, lidocaine.

VR1 agonists and antagonists such as e.g. NGX-4010, WL-1002, ALGRX-4975, WL-10001, AMG-517.

Nicotine receptor agonists such as e.g. ABT-202, A-366833, ABT-594, BTG-102, A-85380, CGX1204.

P2X3-receptor antagonists such as e.g. A-317491, ISIS-13920, AZD-9056.

NGF agonists and antagonists such as e.g. RI-724, RI-1024, AMG-819, AMG-403, PPH 207.

NK1 and NK2 antagonists such as e.g. DA-5018, R-116301, CP-728663, ZD-2249.

NMDA antagonists such as e.g. NER-MD-11, CNS-5161, EAA-090, AZ-756, CNP-3381. potassium channel modulators such as e.g. CL-888, ICA-69673, retigabin.

GABA modulators such as e.g. lacosamide.

Serotonergic and noradrenergic modulators such as e.g. SR-57746, paroxetine, duloxetine, clonidine, amitriptyline, citalopram, flibanserine.

Anti-migraine drugs such as e.g. sumatriptan, zolmitriptan, naratriptan, eletriptan.

The dosage necessary for obtaining a pain-alleviating effect is, in the case of intravenous administration, expediently from 0.01 to 3 mg/kg of body weight, preferably from 0.1 to 1 mg/kg, and, in the case of oral administration, from 0.1 to 8 mg/kg of body weight, preferably from 0.5 to 3 mg/kg, in each case 1 to 3 times per day. The compounds prepared according to the invention can be administered intravenously, subcutaneously, intramuscularly, intrarectally, intranasally, by inhalation, transdermally or orally, aerosol formulations being particularly suitable for inhalation. They can be incorporated into customary pharmaceutical preparations, such as tablets, coated tablets, capsules, powders, suspensions, solutions, metered-dose aerosols or suppositories, if appropriate together with one or more customary inert carriers and/or diluents, for example with maize starch, lactose, cane sugar, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances, such as hardened fat, or suitable mixtures thereof.

### Experimental Section

Generally, there are IR, ¹H NMR and/or mass spectra for the compounds that were prepared. The ratios given for the eluants are in volume units of the solvents in question. For ammonia, the given volume units are based on a concentrated solution of ammonia in water.

Unless indicated otherwise, the acid, base and salt solutions used for working up the reaction solutions are aqueous systems having the stated concentrations.

For chromatographic purification, silica gel from Millipore (MATREX™, 35-70 μm) or Alox (E. Merck, Darmstadt, Alumina 90 standardized, 63-200 μm, article No. 1.01097.9050) are used.

In the descriptions of the experiments, the following abbreviations are used:
CDI 1,1′-carbonyldiimidazole
TLC thin layer chromatogram
DIPEA diisopropylethylamine
DMAP 4-dimethylaminopyridine
DMF dimethylformamide
DMSO dimethylsulphoxide
HATU O-(7-azabenzotriazol-1-yl)-N,N,N′,N′-tetramethyluronium hexafluorophosphate
tert tertiary
TBTU 2-(1H-benzotriazol-1-yl)-1.1.3.3-tetramethyluronium-tetrafluoroborate
THF tetrahydrofuran

The following analytical HPLC methods were used:

Method 1

| Column: | Zorbax Stable Bond C18, 3.5 μM, 4.6 × 75 mm | | |
|---|---|---|---|
| Detection: | 230-360 nm | | |
| Eluant A: | water/0.1% formic acid | | |
| Eluant B: | acetonitrile/0.1% formic acid | | |
| Gradient: | | | |
| time in min | % A | % B | flow rate in mL/min |
| 0.0 | 95.0 | 5.0 | 1.6 |
| 0.1 | 95.0 | 5.0 | 1.6 |
| 4.5 | 10.0 | 90.0 | 1.6 |
| 5.09 | 10.0 | 90.0 | 1.6 |
| 5.5 | 90.0 | 10.0 | 1.6 |

Method 2:

| Column: | Interchim Strategy C18, 5 μM, 4.6 × 50 mm | | |
|---|---|---|---|
| Detection: | 220-320 nm | | |
| Eluant A: | water/0.1% TFA | | |
| Eluant B: | acetonitrile | | |
| Gradient: | | | |
| time in min | % A | % B | flow rate in mL/min |
| 0.0 | 95.0 | 5.0 | 3.0 |
| 0.3 | 95.0 | 5.0 | 3.0 |
| 2.0 | 2.0 | 98.0 | 3.0 |
| 2.4 | 2.0 | 98.0 | 3.0 |
| 2.45 | 95.0 | 5.0 | 3.0 |
| 2.8 | 95.0 | 5.0 | 3.0 |

The following HPLC-method was used for the preparative separation of enantiomers:

Method 3:

| | |
|---|---|
| Column: | Daicel OJ-H, 250 × 4.6 mm, 5 μm |
| Detection: | 230-360 nm |
| Eluant: | n-hexane + 0.2% diethylamine/ethanol = 70:30 |
| Flow rate: | 12 ml/min |
| Gradient: | isocratic |

Preparation of the End Compounds

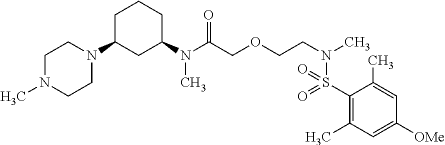

### EXAMPLE 1

1a)

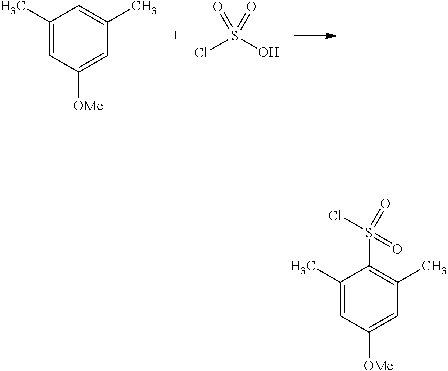

A mixture of 2.0 g (14.69 mmol) 3,5-dimethylanisol and 20 ml dichloromethane was combined with 5.85 ml (88.0 mmol) chlorosulphonic acid while cooling with an ice bath. The reaction mixture was then stirred for 20 min at ambient temperature and then poured onto 50 ml ice water. The mixture was extracted with 100 ml dichloromethane. The organic extracts were washed with 5% sodium hydrogen carbonate solution, dried on sodium sulphate and evaporated to dryness.

C₉H₁₁ClO₃S (234.70)

[M+H]+=234/236

TLC: silica gel, petroleum ether/ethyl acetate 9:1, Rf value=0.46

1b)

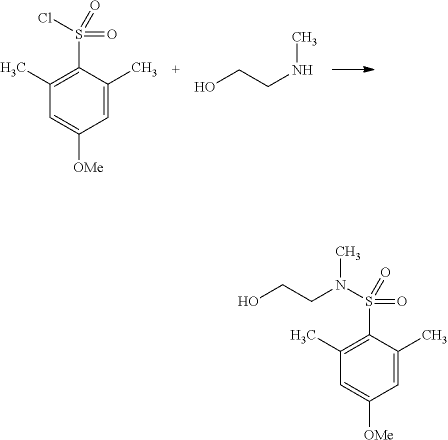

1.69 g (21.1 mmol) N-methylaminoethanol (BASF) and 6.68 ml (47.9 mmol) triethylamine were dissolved in 100 ml dichloromethane. At 0° C., 4.50 g (19.2 mmol) product of 1a dissolved in 50 ml dichloromethane were added dropwise. The cooling was removed and the mixture was stirred for 1.5 hours at ambient temperature. The reaction mixture was then washed with 1 N hydrochloric acid and 5% sodium hydrogen carbonate solution. The organic phase was dried on sodium sulphate and evaporated to dryness.

C₁₂H₁₉NO₄S (273.35)

[M+H]+=274

TLC: silica gel, dichloromethane/ethanol 19:1, Rf value=0.43

1c)

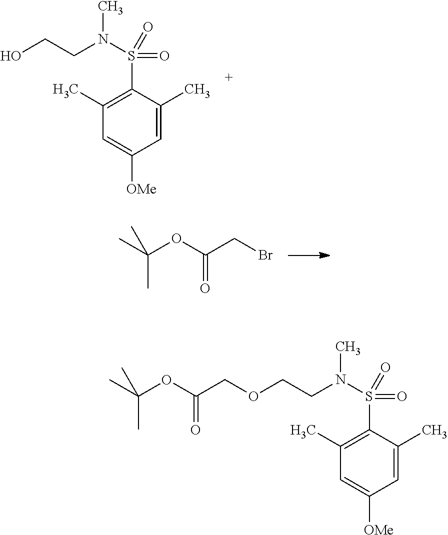

A mixture of 5.15 g (18.8 mmol) product from 1b), 1.75 g (6.60 mmol) tetrabutylammonium chloride (Fluke) and 80 ml of toluene was combined first with 100 ml of 35% sodium hydroxide solution, then with 4.18 ml (28.3 mmol) tert-butyl bromoacetate in 20 ml of toluene at 0° C. The reaction mixture was then stirred for 1.5 hours at ambient temperature, then diluted with diethyl ether. After the phase separation the organic phase was washed four times with water until neutral, dried on sodium sulphate and evaporated to dryness in vacuo. The crude product thus obtained was purified by column chromatography through silica gel (eluant: petroleum ether/ethyl acetate 4:1).

C₁₈H₂₉NO₆S (387.49)

[M+H]+=388

TLC: silica gel, petroleum ether/ethyl acetate 7:3, Rf value=0.59

1d)

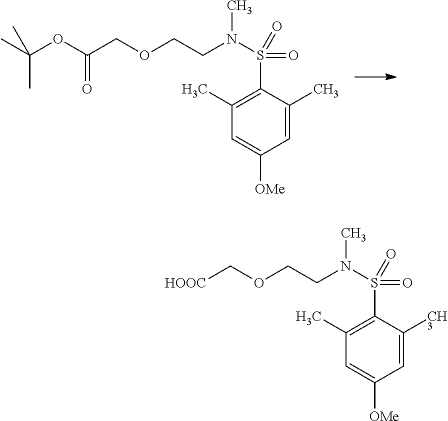

A mixture of 6.80 g (17.6 mmol) of product from 1c), 8 ml TFA and 100 ml dichloromethane was stirred for 2.5 hours at ambient temperature. The reaction mixture was then evaporated to dryness in vacuo. The residue was combined with 1 N sodium hydroxide solution and extracted twice with ethyl acetate (the organic extracts were discarded). The aqueous phase was acidified with 2 M hydrochloric acid, then extracted again with ethyl acetate. The organic extracts were washed with water, dried on sodium sulphate and evaporated to dryness in vacuo.

C₁₄H₂₁NO₆S (331.29)

[M+H]+=332

HPLC: retention time=3.4 min

1e)

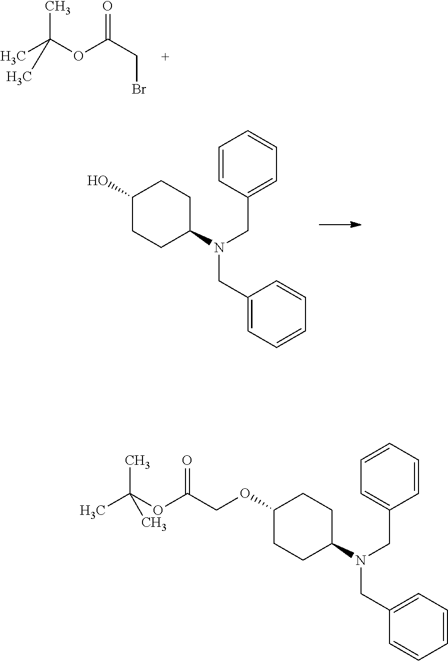

100 g (338 mmol) trans-4-dibenzylamino-cyclohexanol [Tetrahedron Lett. 36 (1995) 1709] were dissolved in 1 l toluene and mixed with 4.2 g (10 mmol) of tetrabutylammonium hydrogen sulphate and 92 ml (508 mmol) tert-butyl bromoacetate. 330 ml of 50% sodium hydroxide solution were added dropwise with vigorous stirring. The mixture was stirred for 18 hours at ambient temperature. After phase separation the organic phase was washed with water and saturated saline solution, dried on magnesium sulphate and then evaporated down in vacuo. The crude product thus obtained was chromatographed on silica gel (cyclohexane/ethyl acetate=4:1) and then recrystallised from petroleum ether.

C₂₆H₃₅NO₃ (409.56)

melting point: 86° C.

[M+H]+=410

TLC: silica gel, cyclohexane/ethyl acetate 4:1, Rf value=0.58

1f)

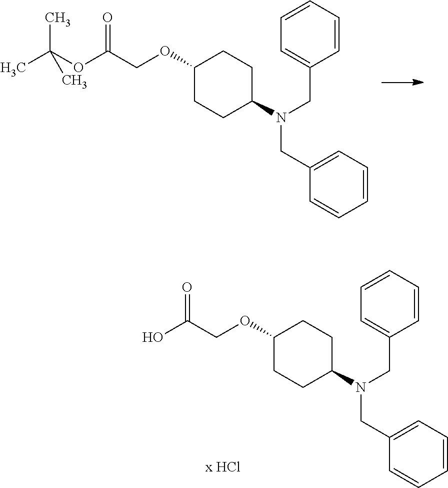

2.0 g (4.88 mmol) product 1e) were dissolved in 5 ml dichloromethane and mixed with 5 ml trifluoroacetic acid. The mixture was stirred for 1 hour at ambient temperature and then the solvent was eliminated in vacuo. The residue was dissolved in 5 ml dioxane and combined with 2 ml of 4N hydrochloric acid in dioxane with stirring. 80 ml ether were added with cooling. The precipitate was suction filtered and dried in the desiccator.

C₂₂H₂₇NO₃×HCl (389.92)

[M+H]+=354

1g)

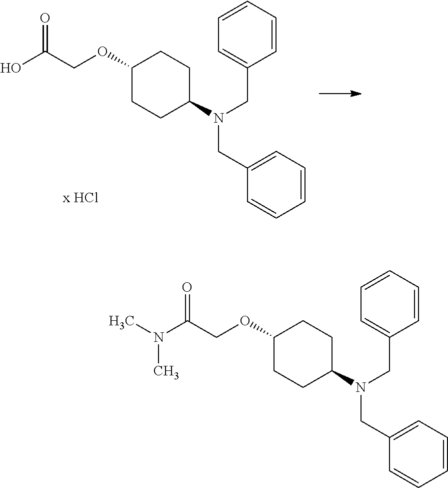

700 mg (1.80 mmol) of product 1f) and 0.3 ml (2.15 mmol) of triethylamine were dissolved in 7 ml THF and mixed at ambient temperature with 320 mg (1.98 mmol) of CDl. The mixture was stirred for 15 minutes at ambient temperature and then 1.80 ml (3.60 mmol) of 2M dimethylamine solution in THF was added. After 2 hours' stirring at ambient temperature the solvent was eliminated in vacuo. The residue was taken up in ethyl acetate and washed with 0.5M potassium hydrogen sulphate solution. The organic phase was dried and evaporated down in vacuo. The crude product thus obtained was reacted further without purification.

C₂₄H₃₂N₂O₂ (380.52)

[M+H]+=381

1h)

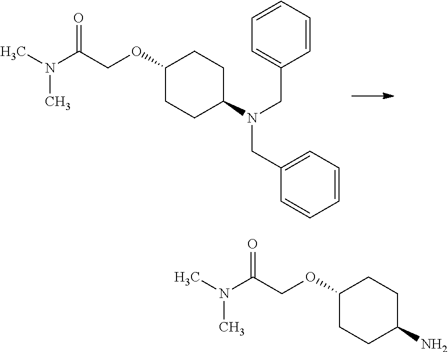

215 mg (0.56 mmol) crude product 1g) were dissolved in 10 ml of methanol and hydrogenated on 50 mg of palladium hydroxide catalyst under a hydrogen pressure of 50 psi. The crude product obtained after elimination of the solvent was reacted further without purification.

C₁₀H₂₀N₂O₂ (200.28)

[M+H]+=201

1i)

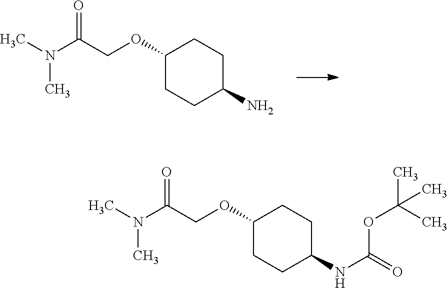

105 mg (0.53 mmol) crude product 1h) and 88 μl (0.63 mmol) triethylamine were dissolved in 5 ml dichloromethane and mixed with 55 μl (0.58 mmol) ethyl chloroformate. The mixture was stirred for 30 minutes at ambient temperature. The reaction solution was diluted with 20 ml dichloromethane and washed with 0. M potassium hydrogen sulphate solution. The organic phase was dried and evaporated down in vacuo. The crude product thus obtained was reacted further without purification.

C₁₃H₂₄N₂O₄ (272.34)

[M+H]+=273

1j)

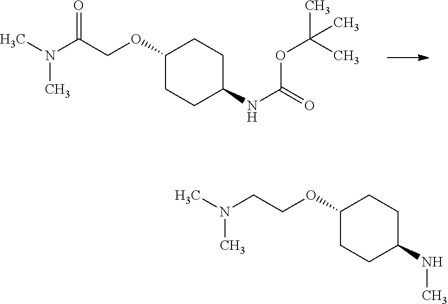

5 ml (10 mmol) of a 2M lithium aluminium hydride solution in THF were refluxed and combined with a solution of 105 mg (0.39 mmol) crude product 1i) in 3 ml THF. The mixture was refluxed for 1 hour. Then 5 ml of water and 1 ml of 2N sodium hydroxide solution were added while the mixture was cooled. The reaction solution was filtered through Celite. The filtrate was freed from the solvent in vacuo and reacted further without purification.

C₁₁H₂₄N₂O (200.32)

[M+H]+=201

1k)

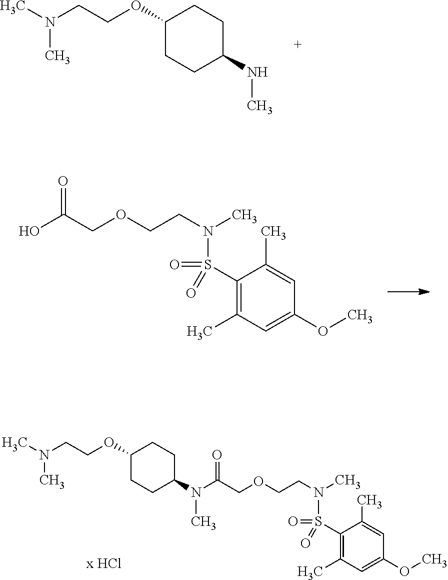

106 mg (0.32 mmol) product 1d), 67 μl (0.48 mmol) triethylamine and 123 mg (0.38 mmol) TBTU were dissolved in 5 ml DMF. After 5 minutes stirring at ambient temperature 80 mg (0.4 mmol) of product 1j) were added. The mixture was stirred for 2 hours at ambient temperature. The residue obtained after elimination of the solvent in vacuo was subjected to reverse phase chromatography (Intgerchim Strategy C18) (acetonitrile-water gradient). The product thus obtained was mixed with ammonia and extracted with dichloromethane. The organic phase was dried and evaporated down in vacuo. The residue was taken up in dioxane and combined with 0.1 ml of 4M hydrochloric acid. After elimination of the solvent in vacuo the product was obtained as the hydrochloride salt.

C₂₅H₄₃N₃O₆S×HCl (513.70)

[M+H]⁺=514

analytical HPLC (method 1); retention time: 1.34 min

The following substances were prepared analogously:

### EXAMPLE 2

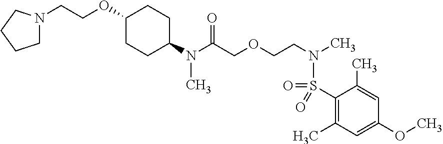

C₂₇H₄₅N₃O₆S (539.73)

[M+H]⁺=540

Analytical HPLC (method 1); retention time: 1.37 min

### EXAMPLE 3

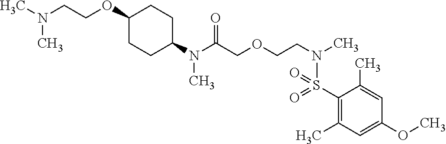

3a)

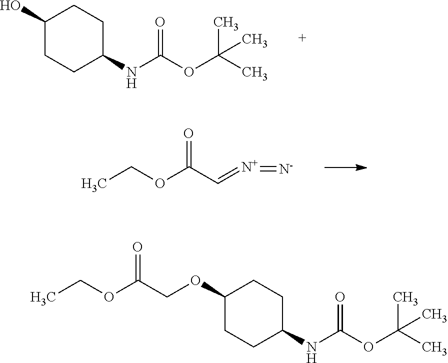

41.3 mg rhodium(II)-acetate (Dimer, Aldrich) were added to a solution of 2.013 g (9.35 mmol) of trans-4-(tert-butyloxycarbonylamino)-cyclohexanol in 20 ml dichloromethane and 1.16 ml (11.2 mmol) of ethyl diazo-acetate, dissolved in 5 ml dichloromethane, was added dropwise with stirring. The mixture was stirred further overnight at ambient temperature, then diluted with 30 ml dichloromethane and washed with 20 ml of semi-concentrated sodium hydrogen carbonate solution. The organic phase was dried and evaporated down and the crude product thus obtained was chromatographed on silica gel.

Yield: 71% of theory

C₁₅H₂₇NO₅ (301.38)

[M+H]⁺=302

3b)

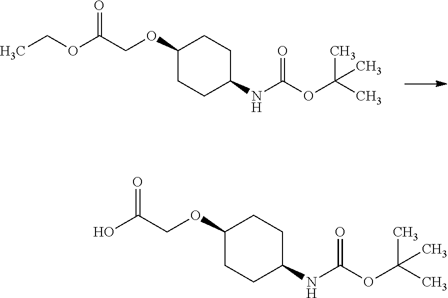

2.00 g (6.64 mmol) of the product from 3a) were hydrolysed under standard conditions in a mixture of sodium hydroxide solution and ethanol.

Yield: 99% of theory

C₁₃H₂₃NO₅ (273.33)

[M+H]⁺=274

3c)

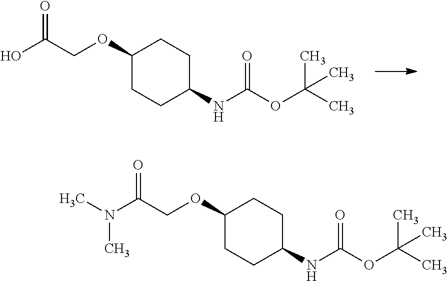

The product from 3b (892 mg, 3.26 mmol) was derivatised with dimethylamine under standard conditions (TBTU, triethylamine in DMF, 2 hours at ambient temperature) to obtain the dimethylamide. The product thus obtained was reacted further without purification.

Yield: 89% of theory

C₁₅H₂₈N₂O₄ (300.39)

[M+H]⁺=301

3d)

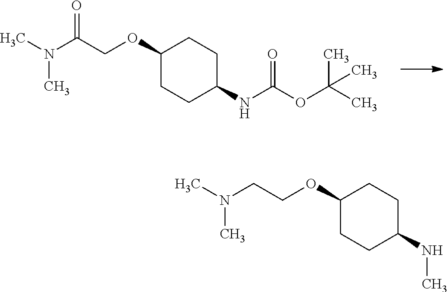

Analogously to Example 1j) the product from 3c (880 mg, 2.93 mmol) was reduced with lithium aluminium hydride in THF to form the diamine.

Yield: 76% of theory

C₁₁H₂₄N₂O (200.32)

[M+H]⁺=201

3e)

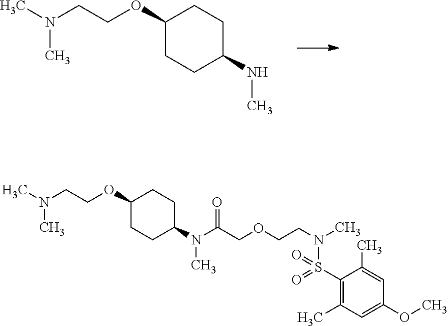

Analogously to Example 1k) the product from 3d) (150 mg, 0.45 mmol) was reacted with the carboxylic acid from 1d).

Yield: 69% of theory

C₂₅H₄₃N₃O₆S (513.70)

[M+H]⁺=514

Analytical HPLC (method 1); retention time: 1.36 min

### EXAMPLE 4

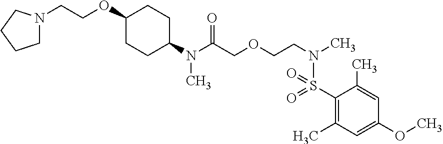

C₂₇H₄₅N₃O₆S (539.73)

[M+H]⁺=540

Analytical HPLC (method 1); retention time: 1.38 min

### EXAMPLE 5

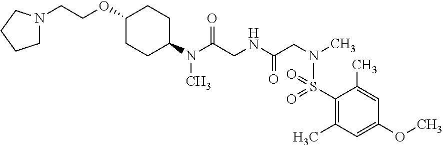

C₂₇H₄₄N₄O₆S (552.73)

[M+H]⁺=553

Analytical HPLC (method 1); retention time: 1.35 min

### EXAMPLE 6

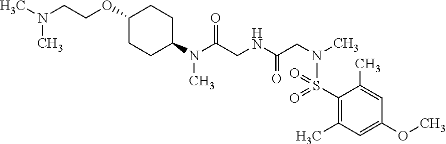

C₂₅H₄₂N₄O₆S (526.69)

[M+H]⁺=527

Analytical HPLC (method 1); retention time: 1.38 min

### EXAMPLE 7

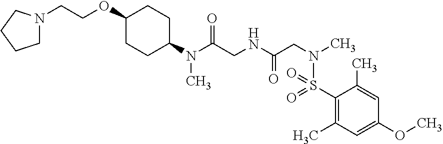

C₂₇H₄₄N₄O₆S (552.73)

[M+H]⁺=553

Analytical HPLC (method 1); retention time: 1.35 min

### EXAMPLE 8

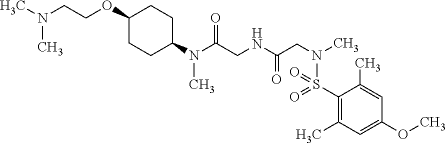

C₂₅H₄₂N₄O₆S (526.69)

[M+H]⁺=527

Analytical HPLC (method 1); retention time: 1.32 min

### EXAMPLE 9

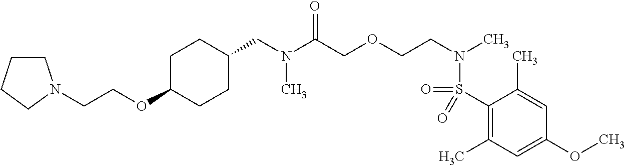

C₂₈H₄₇N₃O₆S (553.76)

[M+H]⁺=554

Analytical HPLC (method 2); retention time: 1.56 min

### EXAMPLE 10

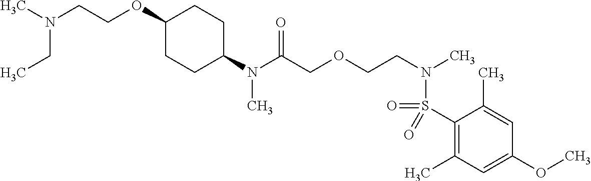

C₂₆H₄₅N₃O₆S (527.72)

[M+H]⁺=528

Analytical HPLC (method 1); retention time: 1.39 min

### EXAMPLE 11

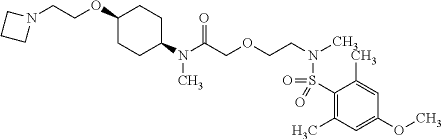

C₂₆H₄₃N₃O₆S (525.71)

[M+H]⁺=526

Analytical HPLC (method 1); retention time: 1.39 min

### EXAMPLE 12

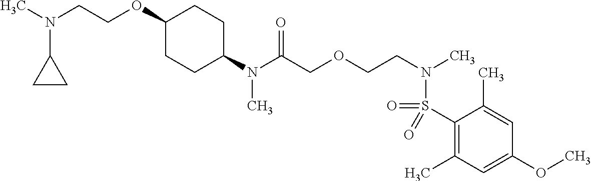

C₂₇H₄₅N₃O₆S (539.73)

[M+H]⁺=526

Analytical HPLC (method 1); retention time: 1.40 min

### EXAMPLE 13

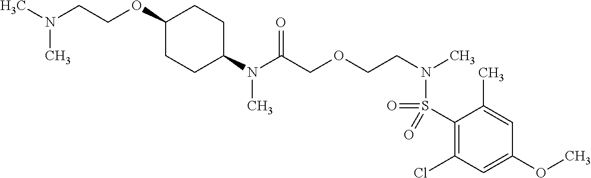

C₂₃H₃₇Cl₂N₃O₆S (554.53)

[M+H]⁺=554/56/58

Analytical HPLC (method 2); retention time: 1.13 min

### EXAMPLE 14

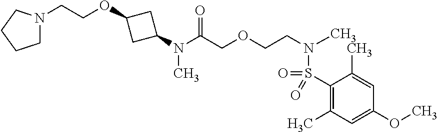

C₂₅H₄₁N₃O₆S (511.68)

[M+H]⁺=512

Analytical HPLC (method 2); retention time: 1.59 min

### EXAMPLE 15

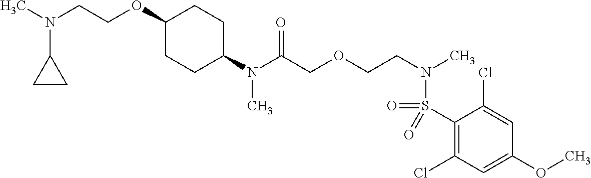

C₂₅H₃₉Cl₂N₃O₆S (580.57)

[M+H]⁺=580/82/84

Analytical HPLC (method 1); retention time: 1.75 min

### EXAMPLE 16

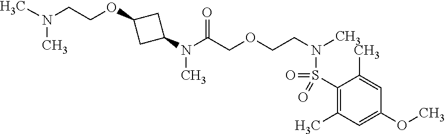

C₂₃H₃₉N₃O₆S (485.64)

[M+H]⁺=486

Analytical HPLC (method 2); retention time: 1.55 min

### EXAMPLE 17

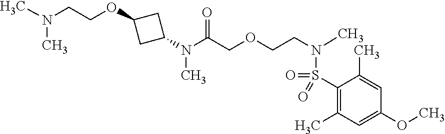

C₂₃H₃₉N₃O₆S (485.64)

[M+H]⁺=486

Analytical HPLC (method 2); retention time: 1.57 min

### EXAMPLE 18

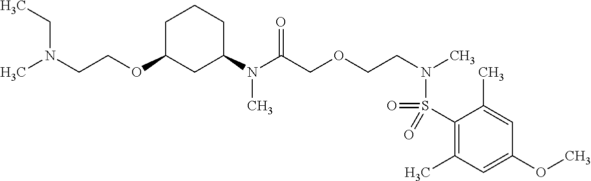

C₂₆H₄₅N₃O₆S (527.72)

[M+H]⁺=528

Analytical HPLC (method 1); retention time: 1.40 min

### EXAMPLE 19

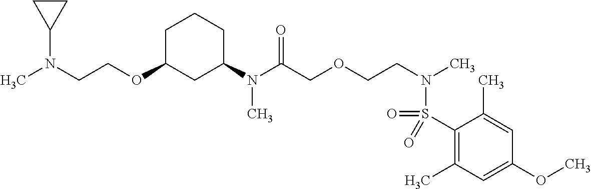

C₂₇H₄₅N₃O₆S (539.73)

[M+H]⁺=540

Analytical HPLC (method 1); retention time: 1.42 min

### EXAMPLE 20

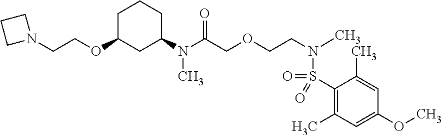

C₂₆H₄₃N₃O₆S (525.71)

[M+H]⁺=526

Analytical HPLC (method 1); retention time: 1.40 min

### EXAMPLE 21

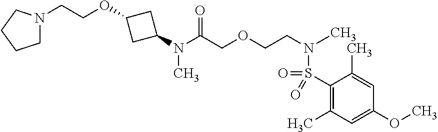

C₂₅H₄₁N₃O₆S (511.68)

[M+H]⁺=512

Analytical HPLC (method 2); retention time: 1.62 min

### EXAMPLE 22

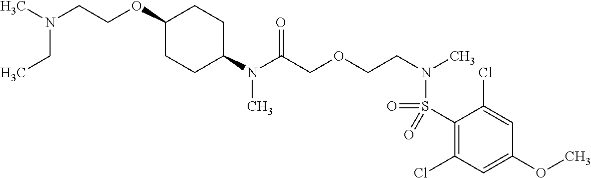

C₂₄H₃₉Cl₂N₃O₆S (568.56)

[M+H]⁺=568/70/72

Analytical HPLC (method 1); retention time: 1.71 min

### EXAMPLE 23

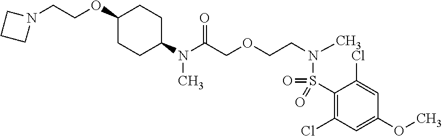

C₂₄H₃₇Cl₂N₃O₆S (566.54)

[M+H]⁺=566/68/70

Analytical HPLC (method 1); retention time: 1.71 min

The following Examples describe pharmaceutical formulations which contain as active substance any desired compound of general formula I:

### EXAMPLE I

Dry Ampoule with 75 mq of Active Compound per 10 ml

Composition:

| | | |
|---|---|---|
| Active compound | 75.0 | mg |
| Mannitol | 50.0 | mg |
| Water for injection | ad 10.0 | ml |

Production:

Active compound and mannitol are dissolved in water. The charged ampoules are freeze dried. Water for injection is used to dissolve to give the solution ready for use.

### EXAMPLE II

Tablet with 50 mq of Active Compound

Composition:

| | | |
|---|---|---|
| (1) Active compound | 50.0 | mg |
| (2) Lactose | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

Production:

(1), (2) and (3) are mixed and granulated with an aqueous solution of (4). (5) is admixed to the dry granules. Tablets are compressed from this mixture, biplanar with a bevel on both sides and dividing groove on one side.

Diameter of the tablets: 9 mm.

### EXAMPLE Ill

Tablet with 350 mq of Active Compound

Composition:

| | | |
|---|---|---|
| (1) Active compound | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

Production:

(1), (2) and (3) are mixed and granulated with an aqueous solution of (4). (5) is admixed to the dry granules. Tablets are compressed from this mixture, biplanar with a bevel on both sides and dividing groove on one side.

Diameter of the tablets: 12 mm.

### EXAMPLE IV

Capsule with 50 mg of Active Compound

Composition:

| | | |
|---|---|---|
| (1) Active compound | 50.0 | mg |
| (2) Maize starch dried | 58.0 | mg |
| (3) Lactose powdered | 50.0 | mg |
| (4) Magnesium stearate | 2.0 | mg |
| | 160.0 | mg |

Production:

(1) is triturated with (3). This trituration is added to the mixture of (2) and (4) with vigorous mixing.

This powder mixture is packed into hard gelatine two-piece capsules of size 3 in a capsule-filling machine.

### EXAMPLE V

Capsules with 350 mg of Active Compound

Composition:

| | | |
|---|---|---|
| (1) Active compound | 350.0 | mg |
| (2) Maize starch dried | 46.0 | mg |
| (3) Lactose powdered | 30.0 | mg |
| (4) Magnesium stearate | 4.0 | mg |
| | 430.0 | mg |

Production:

(1) is triturated with (3). This trituration is added to the mixture of (2) and (4) with vigorous stirring.

This powder mixture is packed into hard gelatine two-piece capsules of size 0 in a capsule-filling machine.

### EXAMPLE VI

Suppositories with 100 mg of Active Compound

1 suppository comprises:

| | | |
|---|---|---|
| Active compound | 100.0 | mg |
| Polyethylene glycol (M.W. 1500) | 600.0 | mg |
| Polyethylene glycol (M.W. 6000) | 460.0 | mg |
| Polyethylene sorbitan monostearate | 840.0 | mg |
| | 2000.0 | mg |

1. A compound of the formula I
wherein 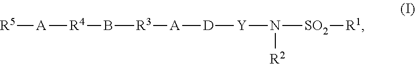
A¹ denotes —CH₂— or a bond,
A² denotes a bond,
B denotes —O—,
D-Y together denote the group 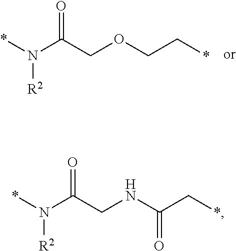
R¹ denotes the group 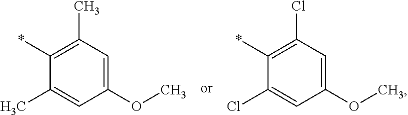
R² denotes H or C₁₋₃-alkyl-, while each methylene group may be substituted by up to two fluorine atoms and each methyl group may be substituted by up to three fluorine atoms, or H₃C—C(O)—,
R³ denotes a C₄₋₆-cycloalkylene group which may be substituted by one, two or three groups R^{3.1},
R^{3.1} denotes —CH₃, —C₂H₅, iso-propyl, tert-butyl, —OH, F, Cl, Br, or I,
R⁴ denotes C₁₋₄-alkylene,
R⁵ denotes H₂N—, C₁₋₄-alkyl-NH—, (C₃₋₆-cycloalkyl)-NH—, (C₁₋₄-alkyl)₂N, (C₁₋₄-alkyl)(C₃₋₆-cycloalkyl)N— or a 4- to 7-membered saturated heterocyclic ring mono- or disubstituted by R^{5.1}, while the groups R^{5.1} in each case may be identical or different, and
R^{5.1} denotes H, F, Cl, Br, I, C₁₋₃-alkyl-, HO—, C₁₋₃-alkyl-O—, (C₁₋₃-alkyl)₂N— or C₁₋₃-alkyl-O—C₂₋₄-alkylene-O—,
or a salt thereof.

2. A compound of the formula I according to claim 1, wherein
R³ denotes a C₄₋₆-cycloalkylene group which may be substituted by one, two or three groups R^{3.1}, and
R^{3.1} denotes —CH₃, —C₂H₅, iso-propyl, tert-butyl, —OH, F, Cl, Br or I,
with the proviso that the above-mentioned C₄₋₆-cycloalkylene group is linked to the remaining molecule in the 1,3 position,
or a salt thereof.

3. A compound of the formula I according to claim 1, wherein
R¹ denotes the group 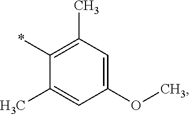
or a salt thereof.

4. A compound of the formula I according to claim 1, wherein
R¹ denotes the group 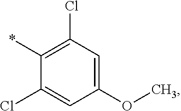
or a salt thereof.

5. A compound of the formula I according to claim 1, wherein
R⁴ denotes —CH₂—CH₂—,
or a salt thereof.

6. A compound of the formula I according to claim 1, wherein
R⁵ denotes the group selected from 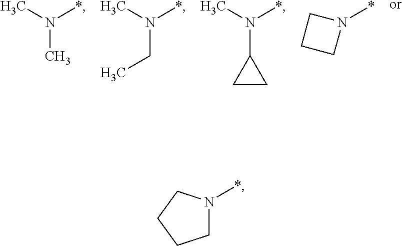
or a salt thereof.

7. A compound of the formula I according to claim 1, wherein
A¹ denotes —CH₂— or a bond,
A² denotes a bond,
B denotes —O—,
D-Y together denote the group 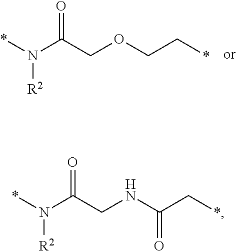
R¹ denotes the group 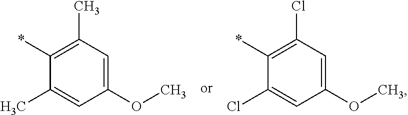
R² denotes H or C₁₋₃-alkyl-, wherein each methylene group may be substituted by up to two fluorine atoms and each methyl group may be substituted by up to three fluorine atoms, or also H₃C—C(O)—,
R³ denotes a C₄₋₆-cycloalkylene group,
R⁴ denotes —CH₂—CH₂— and
R⁵ denotes the group 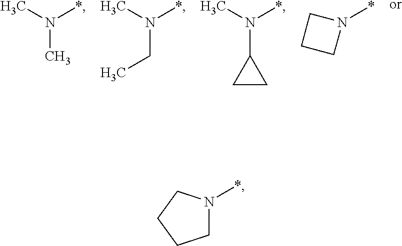
or a salt thereof.

8. A compound of the formula I according to claim 1 selected from the group consisting of:

| Example | Structure |
|---|---|
| (1) | 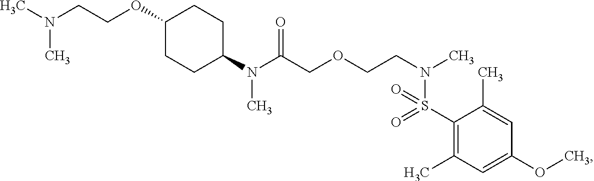 |
| (2) | 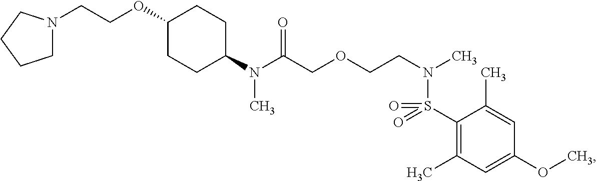 |
| (3) | 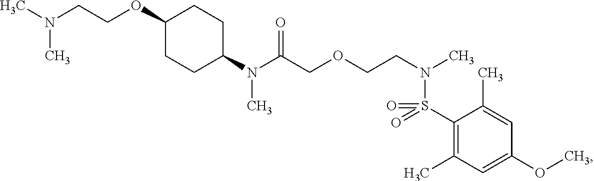 |
| (4) | 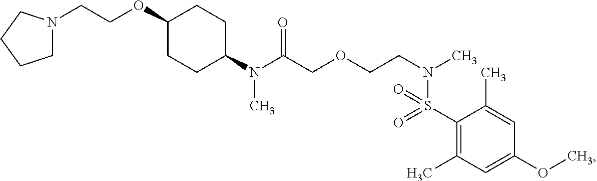 |
| (5) | 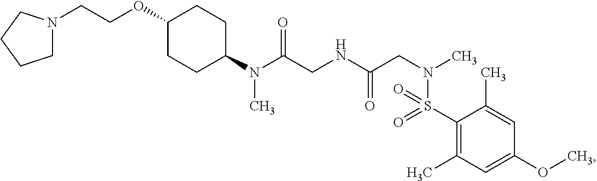 |
| (6) | 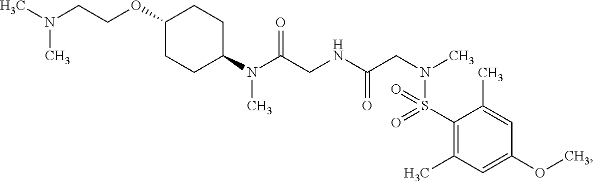 |
| (7) | 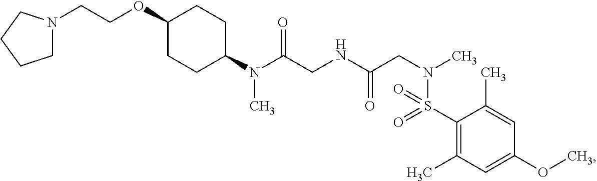 |
| (8) | 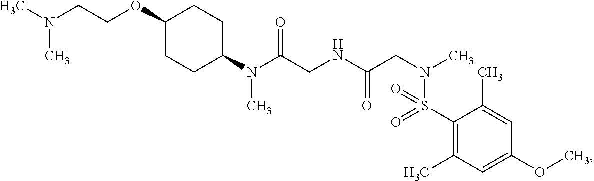 |
| (9) | 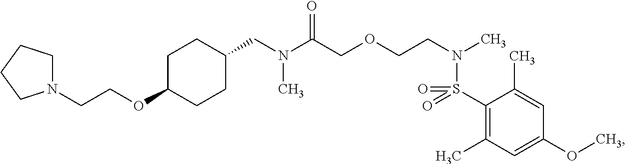 |
| (10) | 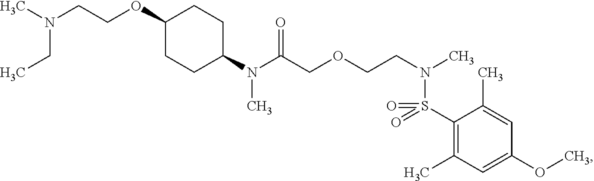 |
| (11) | 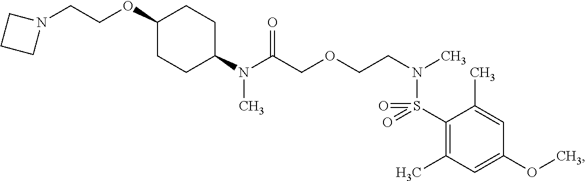 |
| (12) | 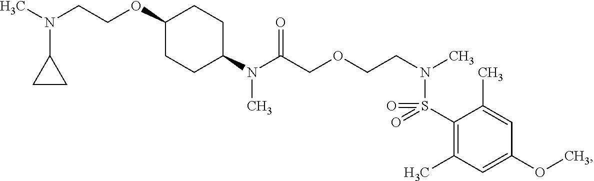 |
| (13) | 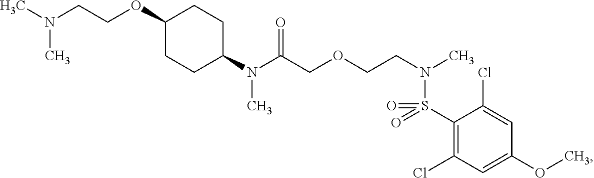 |
| (14) | 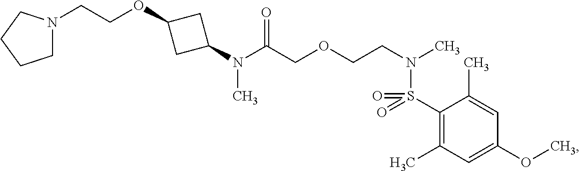 |
| (15) | 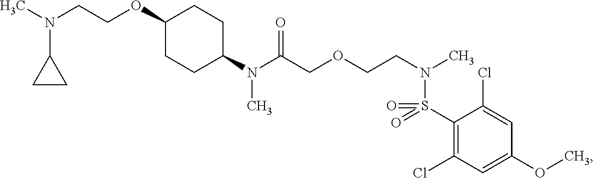 |
| (16) | 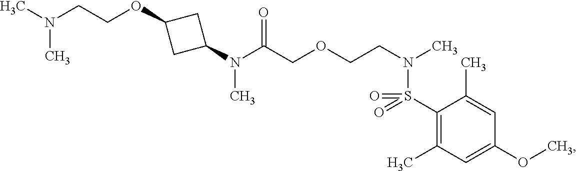 |
| (17) | 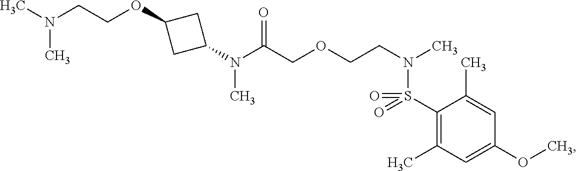 |
| (18) | 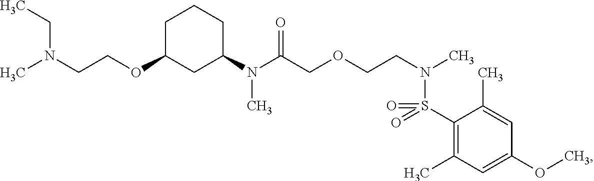 |
| (19) | 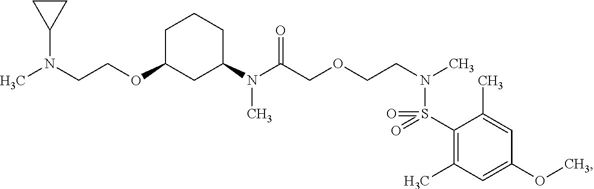 |
| (20) | 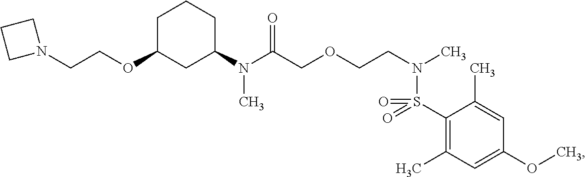 |
| (21) | 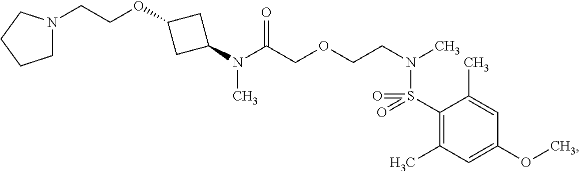 |
| (22) | 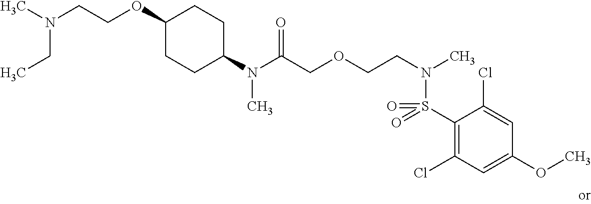 |
| (23) | 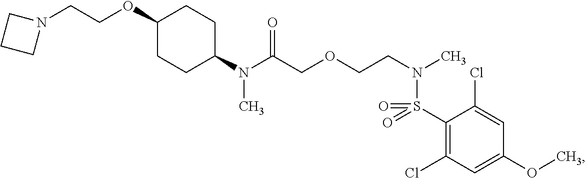 |

or a salt thereof.

9. A physiologically acceptable salt of a compound according to claim 1, 2, 3, 4, 5, 6, 7 or 8.

10. A pharmaceutical composition comprising a compound according to claim 1, 2, 3, 4, 5, 6, 7 or 8 or a physiologically acceptable salt thereof together with a carrier or diluent.

11. A method for treating acute pain, visceral pain, neuropathic pain, inflammatory/pain receptor-mediated pain, tumour pain and headache diseases which comprises administering to a host suffering from the same a therapeutically effective amount of a compound according to claim 1, 2, 3, 4, 5, 6, 7 or 8 or a physiologically acceptable salt thereof

12. (canceled)
